(19) United States
(12) Patent Application Publication
(10) Pub. No.: US 2024/0012010 A1
(43) Pub. Date: Jan. 11, 2024

(54) METHODS OF MONITORING ALPHA-1 ANTITRYPSIN (AAT) DEFICIENCY BY MEASURING POLYMERISED AAT

(71) Applicant: Vertex Pharmaceuticals Incorporated, Boston, MA (US)

(72) Inventors: Marina Simeone PENNEY, Acton, MA (US); Christi D. COOK, Wakefield, MA (US)

(21) Appl. No.: 18/036,491

(22) PCT Filed: Nov. 11, 2021
(86) PCT No.: PCT/US2021/072352

§ 371 Date: May 11, 2023

Related U.S. Application Data
(60) Provisional application No. 63/112,820, filed on Nov. 12, 2020.

(51) Int. Cl. G01N 33/68; A61K 31/4162

(52) U.S. Cl. CPC G01N 33/6893; A61K 31/4162; G01N 2333/8125; G01N 2800/52; G01N 2800/085; G01N 2470/04

(57) ABSTRACT

This application describes methods of measuring AAT polymer and kits for use in the same. Also described herein are methods for monitoring liver disease in AATD patients, methods for measuring AAT polymer in AATD patients, methods for treating AATD patients using an AAT modulator, and methods of measuring the efficacy of AAT modulators.

This application claims the benefit of priority of U.S. Provisional Application No. 63/112,820, filed Nov. 12, 2020, the contents of which are incorporated herein by reference in their entirety.

Disclosed herein are methods for measuring the concentration of circulating polymeric Alpha-1 antitrypsin (AAT) in patients with AAT deficiency (AATD). Also disclosed herein are methods for monitoring the disease state of patients with AATD, including methods of detecting liver damage, an increased risk of liver damage, or polymeric AAT in the liver of patients with AATD. Also disclosed herein are methods of treating AATD using an AAT modulator, and methods of identifying AATD patients with an increased likelihood of responding to treatment with an AAT modulator.

Alpha-1 antitrypsin deficiency is a genetic disorder characterized by low circulating levels of monomeric alpha-1 antitrypsin (AAT). AAT is a protein produced primarily in the liver and secreted into the blood, although other cell types, including lung epithelial cells, monocytes, macrophages, and neutrophils, also produce the protein locally (Bergin, et al., Sci Transl Med. 2014;6(217):217ra1; Geraghty, et al., Am J Respir Crit Care Med. 2014;190(11):1229-42). AAT inhibits several serine proteinases secreted by inflammatory cells (most notably neutrophil elastase, cathepsin G, and proteinase-3) and thus protects organs such as the lung from damage by these proteinases, especially during periods of infection and increased inflammation.

The mutation most commonly associated with AATD involves a substitution of lysine for glutamic acid (E342K) in the SERPINA1 gene that encodes the AAT protein. This mutation, known as the Z mutation, leads to misfolding and polymerization of the translated protein within cells, and causes AAT to not be secreted into the bloodstream. The polymerized Z-AAT protein accumulates in the endoplasmic reticulum (ER) of hepatocytes and can result in neonatal liver disease or progressive liver disease in adulthood that can lead to cirrhosis or liver cancer.

The current standard of care for monitoring liver disease in patients with AATD uses liver biopsies and/or imaging techniques (e.g., computerized tomography (CT) scans, magnetic resonance elastography, and transient elastography). Both strategies suffer from several significant shortcomings, however. Liver biopsies are invasive, time consuming, expensive, and only partially quantitative. Imaging methods, while less invasive and more quantitative than liver biopsies, are also less accurate than biopsies, and still expensive and time-consuming to perform. Imaging methods are also not specific for AAT polymer, and therefore are incapable of distinguishing liver damage caused by AATD from that caused by other environmental factors (e.g., alcoholism).

While AAT polymer ELISA assays have previously been described (see, e.g., Miranda et al., Hepatology 52.3 (2010): 1078-1088; Florie Borel and Christian Mueller (eds.), Alpha-1 Antitrypsin Deficiency: Methods and Protocols, Methods in Molecular Biology, vol. 1639 (2017)), the quantitative accuracy and sensitivity of these assays has been limited by reference calibrators that are not reproducible, not quantified for AAT protein content, and not characterized as having a polymer size distribution that is representative of AAT polymer sizes found in human patients. Illustratively, PiZ mouse blood (plasma/serum) is commonly used as a reference calibrator for AAT polymer ELISA assays. However, PiZ mice have multiple copies (in some cases up to 16 copies) of the human Z-AAT gene randomly inserted into the genome. This causes higher levels of AAT to be expressed, resulting in higher total protein levels compared to human AATD patients. Moreover, polymers form when mutated protein misfolds and aggregates. Since the mouse has more copies of mutated gene than humans, which only have 2 copies, misfolding and aggregation causes increased polymer formation and larger polymer chain lengths that are not representative of the overall size distribution in human blood.

Consequently, there exists a need in the art for improved tools and methods for monitoring and treating liver damage in patients with AATD. The current disclosure aids in fulfilling these needs. Disclosed herein are immunoassays that exhibit improved reliability, sensitivity, specificity, and accuracy, and exhibit a broader dynamic range than previously disclosed assays for measuring concentrations of circulating AAT polymer. Because of these improvements, the immunoassays disclosed herein enable, for the first time, a biomarker-based assay for monitoring and treating liver disease in patients with AATD. The methods and tools disclosed herein can also be used in combination with AAT modulators to provide improved methods of treatments.

### SUMMARY

In some embodiments, the disclosure provides a method for measuring the concentration of polymerized alpha-1 antitrypsin (AAT) in a patient, wherein the method comprises: (i) using a first antibody to separate polymerized AAT from monomeric AAT in a patient sample, wherein the first antibody binds polymeric AAT with higher affinity than monomeric AAT; (ii) incubating the separated polymerized AAT with a second detection antibody, where the second antibody binds the first antibody-AAT complex; and (iii) measuring the amount of second antibody. In some embodiments, the patient has AATD. In some embodiments, the patient has a Z mutation in the AAT protein. In some embodiments, the AAT polymer is circulating AAT polymer. In some embodiments, the sample is a blood, serum, or plasma sample. In some embodiments, the method is an immunoassay. In some embodiments, the first antibody is 2C1. In some embodiments, the first antibody is ATZ11. In some embodiments, the first antibody is LG96, MG97, or an antigen-binding fragment thereof. In some embodiments, the second antibody is A80-122P.

In some embodiments, the present disclosure provides a kit for measuring the concentration of polymerized alpha-1 antitrypsin (AAT) in a patient, wherein the kit comprises a first antibody that binds polymerized AAT with a higher affinity than monomeric AAT, and a second antibody that binds both monomeric and polymeric AAT. In some embodiments, the first antibody is 2C1. In some embodiments, the first antibody is ATZ11. In some embodiments, the first antibody is LG96, MG97, or an antigen-binding fragment thereof. In some embodiments, the second antibody is A80-122P.

In some embodiments, the present disclosure provides a method of detecting liver damage or an increased risk of liver damage in a patient with alpha-1 antitrypsin deficiency (AATD), wherein the method comprises measuring the concentration of circulating polymerized alpha-1 antitrypsin (AAT) in the patient. In some embodiments, the patient has a Z mutation in the AAT protein. In some embodiments, the patient is heterozygous for the Z mutation and has an additional mutation in AAT associated with AATD. In some embodiments, the patient is homozygous for the Z mutation. In some embodiments, the concentration of circulating polymeric AAT is measured using any one of the methods disclosed herein. In some embodiments, the patient has been administered an AAT modulator or is being considered for treatment with an AAT modulator.

In some embodiments, the present disclosure provides a method of detecting polymeric AAT in the liver of a patient with alpha-1 antitrypsin deficiency (AATD), wherein the method comprises measuring the concentration of circulating polymerized alpha-1 antitrypsin (AAT) in the patient. In some embodiments, the patient has a Z mutation in the AAT protein. In some embodiments, the concentration of circulating polymeric AAT is measured using any one of the methods disclosed herein. In some embodiments, the patient has been administered an AAT modulator, or is being considered for treatment with an AAT modulator.

Also disclosed herein is a method of identifying a patient with alpha-1 antitrypsin deficiency (AATD) that has an increased likelihood of responding to treatment with an AAT modulator, wherein the method comprises measuring the concentration of circulating polymerized alpha-1 antitrypsin (AAT) in the patient. In some embodiments, the patient has a Z mutation in the AAT protein. In some embodiments, the concentration of circulating AAT is measured using any one of the methods disclosed herein.

Also disclosed herein is a method of treating a patient with alpha-1 antitrypsin deficiency (AATD), wherein the method comprises: (i) measuring the concentration of circulating polymerized alpha-1 antitrypsin (AAT) in a patient; and (ii) if the concentration of circulating polymerized AAT is found to be at least 1 μg/mL, administering an AAT modulator to the patient, and if the concentration of circulating polymerized AAT is found to be less than 1 μg/mL, not administering an AAT modulator to the patient. In some embodiments, the concentration of circulating polymerized AAT is determined using any one of the methods disclosed herein.

Also disclosed herein is a method for measuring the efficacy of an alpha-1 antitrypsin (AAT) modulator, wherein the method comprises: (i) administering the AAT modulator to a patient with alpha-1 antitrypsin deficiency (AATD); and (ii) measuring the change in the concentration of circulating polymerized AAT in the patient.

Also disclosed herein is a method for determining an efficacious dosing regimen of an alpha-1 antitrypsin (AAT) modulator for treating alpha-1 antitrypsin deficiency (AATD), wherein the method comprises measuring the change in the concentration of circulating polymerized AAT in the patient.

### BRIEF DESCRIPTION OF THE DRAWINGS

FIGS. 1A-1F depict native western blots and size exclusion chromatograms of plasma purified alpha-1 antitrypsin (M-AAT) treated at 60° C. Monomers, dimers, trimers, and oligomers are indicated on the blots. FIG. 1A and FIG. 1B depict native western blots of M-AAT after 0-24 hours of heat treatment, where the blot has been probed with either a total AAT antibody (FIG. 1A) or an AAT polymer specific antibody (FIG. 1B). FIG. 1C and FIG. 1D depict native western blots of M-AAT after 0-6 hours of heat treatment, where the blots have been probed with either a total AAT primary antibody (FIG. 1C) or an AAT polymer specific primary antibody (FIG. 1D). FIG. 1E and FIG. 1F depict size exclusion chromatograms of M-AAT after 0-24 hours and 2-6 hours of heat treatment, respectively.

FIG. 2 is a representation of a sandwich ELISA method that utilizes a plate coated with capture antibody to first capture and separate the total pool of AAT polymers from monomeric AAT. FIG. 2 is included for illustrative purposes and does not show all possible polymer sizes and interactions with capture/detection antibodies.

FIGS. 3A-3B depict the detection of AAT polymer using an ELISA method in PiZ hemizygous mouse urine, bronchoalveolar lavage fluid (BALF), and plasma (FIG. 3A) and across multiple tissues collected after whole body perfusion (FIG. 3B).

FIG. 4 depicts the concentration of AAT polymer measured by ELISA in human serum and plasma samples (diluted 25,000- or 50,000-fold).

FIGS. 5A-5D depict the results of a 72 hour PiZ mouse study (FIGS. 5A, 5C) and a 28 day PiZ mouse study (FIGS. 5B, 5D) in which eleven week old male PiZ mice, hemizygous for the Z variant of the human allele of AAT, were orally administered a suspension of either vehicle or Compound 2 QD at 50 and 100 mg/kg/day for 72 hours or 5, 16, 50, and 100 mg/kg/day for 28 days. Simple linear regression analysis demonstrated a strong correlation between levels of polymer in PiZ mouse plasma with liver polymer after (FIG. 5C) 72 hours Pearson r=0.94 (95% CI: 0.75, 0.99) or (FIG. 5D) 28 days Pearson r=0.82 (95% CI: 0.59-0.93) of AAT modulator treatment. Liver polymer staining was assessed as the % area of liver tissue that stains positively by monoclonal antibody 2C1 immunohistochemistry.

FIG. 6 depicts the percent recovery for each dilution within the analytical assay range (50-25,000 pg/mL) measured in an assessment of the concentration-response of AAT polymer to the calibration curve in individual donor MZ serum and ZZ plasma samples.

FIG. 7 depicts the results of specificity testing performed to assess the ability of an assay of the present disclosure to detect AAT polymer in the presence of potentially interfering analytes (i.e., serum AAT assumed to be monomeric AAT in PiMM samples).

### DETAILED DESCRIPTION

The following detailed description and examples illustrate certain embodiments of the present disclosure. Those of skill in the art will recognize that there are numerous variations and modifications of this disclosure that are encompassed by its scope. Accordingly, the description of certain embodiments should not be deemed to limit the scope of the present disclosure.

In order that the disclosure may be more readily understood, certain terms are defined throughout the detailed description. Unless defined otherwise herein, all scientific and technical terms used in connection with the present disclosure have the same meaning as commonly understood by those of ordinary skill in the art.

All references cited herein, including, but not limited to, published and unpublished patent applications, granted patents, and literature references, are incorporated herein by reference and are hereby made a part of this specification. To the extent a cited reference conflicts with the disclosure herein, the specification shall control.

As used herein, “AAT” and “A1AT” refer to alpha-1 antitrypsin.

As used herein, the term “AATD” refers to alpha-1 antitrypsin deficiency.

As used herein, the term “mutations” can refer to mutations in the SERPINA1 gene (the gene encoding AAT) or the effect of alterations in the gene sequence on the AAT protein. A “SERPINA1 gene mutation” refers to a mutation in the SERPINA1 gene, and an “AAT protein mutation” refers to a mutation that results in an alteration in the amino acid sequence of the AAT protein. A genetic defect or mutation, or a change in the nucleotides in a gene in general, results in a mutation in the AAT protein translated from that gene.

Each of the mutations discussed herein, and their association with AATD, is well known in the art. See, for example, Miranda et al. (2010), Laffranchi et al., Fra et al., Miranda et al. (2017), and Matamala et al., the contents of each of which are incorporated in their entirety by reference herein. The locations of the mutations provided herein are in reference to the AAT protein lacking the 24-amino acid signaling peptide.

As used herein, a patient who is “homozygous” for a particular gene mutation has the same mutation on each allele.

As used herein, a patient who is “heterozygous” for a particular gene mutation has the particular mutation on only one allele.

As used herein, a patient who has a mutation is either heterozygous or homozygous for that mutation.

As used herein, a patient who has the PiZZ genotype is a patient who is homozygous for the Z mutation in the AAT protein.

The terms “patient” and “subject” are used interchangeably and refer to an animal, including humans.

As used herein, the terms “treatment,” “treating,” and the like generally mean the improvement of AATD or its symptoms and/or lessening the severity of AATD or its symptoms in a subject.

As used herein, the terms “polymeric AAT,” “polymerized AAT,” and “AAT polymer” are interchangeable and refer to a complex of 2 or more AAT proteins, and encompass AAT dimers, AAT trimers, AAT tetramers, AAT pentamers, and larger oligomers.

As used herein, the term “circulating AAT polymer” refers to AAT polymer that is not in a liver cell. In some embodiments, the circulating AAT polymer is in non-liver tissue. In some embodiments, the circulating AAT polymer is extracellular. In some embodiments, the circulating AAT polymer is in extracellular fluid. In some embodiments, the circulating AAT polymer is in BALF (bronchoalveolar lavage fluid), plasma, serum, sputum, urine, skin tissue, intestine tissue, kidney tissue, lung tissue, and/or blood.

As used herein, “2C1” refers to the 2C1 antibody, which is a mouse mAb antibody that binds AAT polymers but not AAT monomer. 2C1 is available commercially from Hycult Biotech. 2C1 was first identified in Miranda et al., 2010, and has been shown to be capable of recognizing polymers comprising a variety of AAT mutants, including the Z mutant (Miranda et al., 2010), the S (E264V), Mₘₐₗₜₒₙ (F52del), and M_{wurzburg} (P369S) mutants (Laffranchi et al, 2018), the Mₚᵢₛₐ (K259I), E_{Taurisano} (K368E) and Y_{orzinuovi} (P391H) mutants (Fra et al, 2018), the

Trento mutant (E75V) (Miranda et al, 2017), and the PiS, PiS+S14F, I50N, A58D, F227C, and T249A mutants (Matamala et al, 2017).

As used herein, “LG96” refers to the monoclonal antibody LG96. LG96, which is deposited under access number DSM ACC3092 at German Collection of Microorganisms and Cell Cultures, was developed by Candor Biosciences. The use of LG96 as a capture antibody capable of binding Z-AAT protein is described in International Patent Publication No. WO2012/038820.

As used herein, “MG97” refers to the monoclonal antibody MG97. MG97 is deposited under access number DSM ACC3093 at German Collection of Microorganisms and Cell Cultures. The use of MG97 as a capture antibody capable of binding Z-AAT protein is described in International Patent Publication No. WO2012/038820.

As used herein, “ATZ11” refers to the ATZ11 antibody, which is a mouse monoclonal antibody that is specific for polymeric AAT. ATZ11 has been shown capable of binding AAT polymers comprising the Z mutant (see Janciauskiene et al., 2002), and the Siiyama, and Mₘₐₗₜₒₙ mutants (see Janciauskiene et al., 2004), among others.

A80-122P refers to the goat polyclonal anti-AAT antibody sold by Bethyl Laboratories.

“AAT modulator” refers to an entity (e.g., a compound, gene therapy, cell therapy, etc.) that modulates the activity of AAT. In some embodiments, the AAT modulator is any one of the AAT modulators disclosed in PCT application number PCT/US2019/054681 (published as WO2020/081257) or PCT/US2020/032832 (published as WO2020/247160), the entire contents of both of which are incorporated by reference herein. In some embodiments, the AAT modulator is any one of the AAT modulators disclosed in PCT application numbers PCT/US2021/025597 (published as WO2021/203010), PCT/US2021/025591 (published as WO2021/203007), PCT/US2021/025614 (published as WO2021/203023), PCT/US2021/025623 (published as WO2021/203028), PCT/US2021/025616 (published as WO2021/203025), and PCT/US2021/025601 (published as WO2021/203014), the contents of all of which are incorporated by reference herein.

In some embodiments, the AAT modulator is

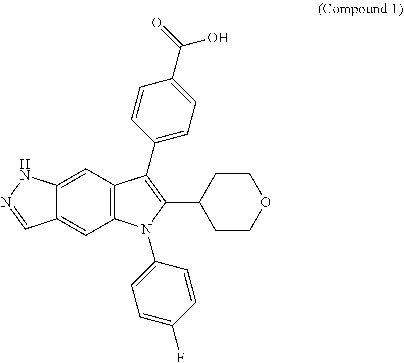 [SMILES: O=C(O)c1ccc(-c2c(C3CCOCC3)n(-c3ccc(F)cc3)c3cc4cn[nH]c4cc23)cc1]

or a pharmaceutically acceptable salt thereof.

In some embodiments, the AAT modulator is

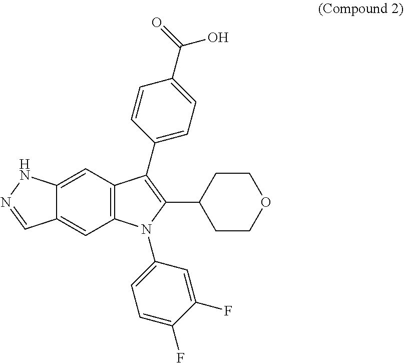 [SMILES: O=C(O)c1ccc(-c2c(C3CCOCC3)n(-c3ccc(F)c(F)c3)c3cc4cn[nH]c4cc23)cc1]

or a pharmaceutically acceptable salt thereof.

As used herein, the terms “about” and “approximately,” when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent. The terms “about” and “approximately” may refer to an acceptable error for a particular value as determined by one of skill in the art, which depends in part on how the values is measured or determined. In some embodiments, the terms “about” and “approximately” mean within 20%, 15%, 10%, 5%, 4%, 3%, 2%, 1%, or 0.5% of a given value or range.

As used herein, the singular forms of a word also include the plural form, unless the context clearly dictates otherwise; as examples, the terms “a,” “an,” and “the” are understood to be singular or plural. By way of example, “an element” means one or more element(s). The term “or” shall mean “and/or” unless the specific context indicates otherwise.

### Methods for Measuring Circulating AAT Polymer Concentrations

The present disclosure provides, in some embodiments, a method for measuring the concentration of polymerized alpha-1 antitrypsin (AAT) in a patient, wherein the method comprises: (i) using a first antibody to separate polymerized AAT from monomeric AAT in a patient sample, wherein the first antibody binds polymeric AAT with higher affinity than monomeric AAT; (ii) incubating the separated polymerized AAT with a second detection antibody, where the second antibody binds the first antibody-AAT complex; and (iii) measuring the amount of second antibody.

The first step of separating the polymerized AAT from monomeric AAT using the first antibody does not require a complete separation of polymerized AAT from monomeric AAT. That is, some AAT monomer may still be present after the first step, so long as the concentration of residual AAT monomer is not high enough to interfere with the subsequent detection steps. In some embodiments, the use of the first antibody increases the concentration of the polymerized AAT relative to the AAT monomer. In some embodiments, the use of the first antibody increases the concentration of AAT polymer relative to the concentration of AAT monomer by approximately ten-fold or higher. In some embodiments, the use of the first antibody increases the concentration of the polymerized AAT relative to the concentration of AAT monomer by approximately 100-fold or higher. In some embodiments, the use of the first antibody increases the concentration of the polymerized AAT relative to the concentration of AAT monomer by approximately 1000-fold or higher. In some embodiments, the concentration of the AAT monomer after use of the first antibody is below the limit of detection of the second antibody.

In some embodiments, the first antibody binds polymeric AAT with a higher affinity than monomeric AAT. In some embodiments, the first antibody binds polymeric AAT with an affinity that is at least 2-fold, at least 5-fold, at least 10-fold, at least 25-fold, at least 50-fold, at least 100-fold, at least 250-fold, at least 500-fold, or at least 1000-fold the affinity with which the first antibody binds monomeric AAT. In some embodiments, the first antibody is 2C1. In some embodiments, the first antibody is ATZ11. In some embodiments, the first antibody is LG96. In some embodiments, the first antibody is MG97.

In some embodiments, the second antibody binds in a location that is compatible with binding of the first antibody (that is, binding of the second antibody does not interfere with binding of the first antibody). In some embodiments, the second antibody binds in a location that is compatible with the binding of 2C1. In some embodiments, the second antibody binds in a location that is compatible with the binding of ATZ11. In some embodiments, the second antibody binds in a location that is compatible with the binding of LG96. In some embodiments, the second antibody binds in a location that is compatible with the binding of MG97.

In some embodiments, the second antibody is A80-122P.

In some embodiments, the patient has alpha-1 antitrypsin deficiency (AATD).

In some embodiments, the patient has a Z mutation (E342K), an S mutation (E264V), a Siiyama mutation (S53F), a Brescia mutation (G22510, an Mₘₐₗₜₒₙ (F52del) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115S) mutation, an X (E204K) mutation, a P_{lowell} (D256V) mutation, or a V333M mutation in the AAT protein.

In some embodiments, the patient has a Z mutation, an S mutation, a Siiyama mutation, a Brescia mutation, an Mₘₐₗₜₒₙ (F52de1) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), PiS+S14F mutations, an I50N mutation, an A58D mutation, an F227C mutation, a T249A mutation, or an H334D mutation in the AAT protein.

In some embodiments, the patient has a Z mutation in the AAT protein. In some embodiments, the patient is heterozygous for the Z mutation. In some embodiments, the patient is heterozygous for the Z mutation, and has an additional AAT mutation associated with AATD. In some embodiments, the additional mutation associated with AATD is an S mutation (E264V), a Siiyama mutation (S531 7), a Brescia mutation (G225R), an Mₘₐₗₜₒₙ (F52del) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115S) mutation, an X (E204K) mutation, a P_{lowell} (D256V) mutation, or a V333M mutation in the AAT protein.

In some embodiments, the patient has a PiZZ genotype. In some embodiments, the patient is homozygous for the S mutation. In some embodiments, the patient is heterozygous for the S mutation. In some embodiments, the patient has an SZ genotype. In some embodiments, the patient is heterozygous for the Z mutation and heterozygous for the Mₘₐₗₜₒₙ (F52del) mutation. In some embodiments, the patient is heterozygous for the Z mutation and heterozygous for the M_{wurzburg} (P369S) mutation. In some embodiments, the patient is heterozygous for the Z mutation and heterozygous for the Mₚᵢₛₐ (K259I) mutation. In some embodiments, the patient is heterozygous for the Z mutation and heterozygous for the E_{Taurisano} (K368E) mutation. In some embodiments, the patient is heterozygous for the Z mutation and heterozygous for the Y_{orzinuovi} (P391H) mutation. In some embodiments, the patient is heterozygous for the Z mutation and heterozygous for the Trento mutation (E75V). In some embodiments, the patient has the PiS+S14F mutation. In some embodiments, the patient is heterozygous for the Z mutation and heterozygous for the I50N mutation. In some embodiments, the patient is heterozygous for the Z mutation and heterozygous for the A58D mutation. In some embodiments, the patient is heterozygous for the Z mutation and heterozygous for the F227C mutation. In some embodiments, the patient is heterozygous for the Z mutation and heterozygous for the T249A mutation.

In some embodiments, the patient sample is a BALF (bronchoalveolar lavage fluid), plasma, serum, sputum, urine, skin, intestine, kidney, liver, lung, or blood sample. In some embodiments, the patient sample is a blood, serum, plasma, urine, or sputum sample. In some embodiments, the patient sample is a blood, serum, or plasma sample. In some embodiments, the patient sample is a blood sample. In some embodiments, the patient sample is a serum sample. In some embodiments, the patient sample is a plasma sample. In some embodiments, the patient sample is a urine sample. In some embodiments, the patient sample is a sputum sample. In some embodiments, the patient sample is a liver sample.

In some embodiments, the AAT polymer is circulating AAT polymer. In some embodiments, the concentration of circulating AAT polymer is measured in a BALF (bronchoalveolar lavage fluid), plasma, serum, sputum, urine, skin, intestine, kidney, liver, lung, or blood sample. In some embodiments, the concentration of circulating AAT polymer is measured in a BALF (bronchoalveolar lavage fluid), plasma, serum, sputum, urine, or blood sample. In some embodiments, the concentration of circulating AAT polymer is measured in a plasma, serum, sputum, urine, or blood sample. In some embodiments, the concentration of circulating AAT polymer is measured in a plasma, serum, urine, or blood sample. In some embodiments, the concentration of circulating AAT polymer is measured in a plasma, serum, or blood sample. In some embodiments, the concentration of circulating AAT polymer is measured in a plasma sample. In some embodiments, the concentration of circulating AAT polymer is measured in a blood sample. In some embodiments, the concentration of circulating AAT polymer is measured in a serum sample.

In some embodiments, the method for measuring the concentration of AAT polymer is an immunoassay. In some embodiments, the immunoassay is an ELISA. In some embodiments, the immunoassay is an immunoprecipitation assay. In some embodiments, the immunoassay is a radioimmunoassay, a chemiluminescent immunoassay, or a fluorescent immunoassay. In some embodiments, the immunoassay is an electrochemiluminescent immunoassay.

In some embodiments, the immunoassay is calibrated using one or more reference standards. In some embodiments, the one or more reference standards comprise heat-treated, plasma-purified M-AAT. In some embodiments, the M-AAT is heat-treated at about 40° C. to about 100° C. In some embodiments, the M-AAT is heat-treated at about 50° C. to about 70° C. In some embodiments, the M-AAT is heat-treated at about 60° C. In some embodiments, the M-AAT is heat-treated for an amount of time sufficient to produce an oligomer distribution that is similar to that of the circulating AAT polymers found in AATD patients. In some embodiments, the M-AAT is heat-treated for an amount of time sufficient to convert at least 90% of the M-AAT into the polymer form. In some embodiments, the M-AAT is heat-treated for an amount of time sufficient to convert at least 95% of the M-AAT into the polymer form. In some embodiments, the M-AAT is heat-treated for at least 1 hour. In some embodiments, the M-AAT is heat-treated for at least 4 hours. In some embodiments, the M-AAT is heat-treated for 1-24 hours. In some embodiments, the M-AAT is heat-treated for 4-18 hours. In some embodiments, the M-AAT is heat-treated for 4-12 hours. In some embodiments, the M-AAT is heat-treated for 4-8 hours. In some embodiments, the M-AAT is heat-treated for 4-6 hours.

The methods disclosed herein provide numerous technical advantages over methods previously used to measure concentrations of circulating AAT polymer in patients. Specifically, the methods disclosed herein are more reliable, more sensitive, more specific, and exhibit a broader dynamic range as compared to previously used methods. Because of these technical advantages, the methods disclosed herein are the first that can be used to monitor liver disease in AATD patients.

Without being bound by theory, the methods disclosed herein produce superior technical results in part because they use a polymer-specific antibody as a capture antibody, and an antibody that binds all forms of AAT as a detection antibody, whereas most previously disclosed methods have used the opposite configuration. Using an antibody that binds all forms of AAT as the capture antibody captures both monomeric and polymeric forms of AAT. The presence of the monomeric AAT may interfere with binding of the polymer-specific antibody during the subsequent detection step. Using the polymer-specific antibody as a capture antibody, in contrast, avoids capture of the interfering monomeric AAT species, which improves the sensitivity, specificity, and dynamic range of the detection method.

Another advantage of the methods disclosed herein is that, unlike previous methods, they can be used in mouse model organisms. Because mice do not naturally suffer from AATD, one common strategy for studying AATD is to use a mouse model organism that comprises human genes encoding AAT with mutations associated AATD. Because these model organisms express both mouse AAT and human AAT, to quantify circulating polymeric AAT it is critical to use a capture antibody and a detection antibody that do not cross react with mouse AAT. Many previously disclosed methods have used detection antibodies that cross react with mouse AAT, and therefore cannot be used to study AATD in mouse model organisms.

The methods disclosed herein are also more reliable and more accurate in part because they are more accurately calibrated than previous methods. As with previous methods, the detection methods disclosed herein are calibrated using heat-treated M-AAT. Heat-treatment causes monomeric M-AAT to polymerize. Producing an AAT polymer distribution that is similar to that found in AATD patients requires careful control of heat-treatment times. Heat-treating for too short a time will not convert enough of the AAT monomers into AAT polymers and will also produce an AAT polymer distribution that is smaller than that found naturally in AATD patients. Heat-treating for too long a time, conversely, will produce an AAT polymer distribution that is larger than that found naturally in AATD patients. Previous methods have been calibrated using AAT reference standards that have been heat-treated for too short a time, and thus comprise a large amount of AAT monomer and AAT polymers that are smaller on average than those found in AATD patients. The methods disclosed herein, in contrast, are calibrated using reference standards that have AAT polymer distributions that are similar to those found naturally in AATD patients. Thus, the methods disclosed herein produce more accurate and more reliable results. In some embodiments, these reference standards are generated by heat-treating plasma-derived AAT at about 60° C. for about 4 hours to about 6 hours.

### Kits for Measuring Circulating AAT Polymer Concentrations

Also disclosed herein are kits for measuring the concentration of circulating polymerized alpha-1 antitrypsin (AAT) in a patient, wherein the kit comprises a first antibody and a second antibody, wherein the first antibody binds polymerized AAT with a higher affinity than monomeric AAT, and wherein the second antibody binds both monomeric and polymeric forms of AAT. In some embodiments, the kit comprises a first antibody that is 2C1 or ATZ11. In some embodiments, the first antibody is 2C1. In some embodiments, the first antibody is ATZ11. In some embodiments, the first antibody is LG96. In some embodiments, the first antibody is MG97. In some embodiments, the kit comprises a second antibody that binds in a location that is compatible with 2C1 binding. In some embodiments, the kit comprises a second antibody that binds in a location that is compatible with ATZ11 binding. In some embodiments, the kit comprises a second antibody that binds in a location that is compatible with LG96 binding. In some embodiments, the kit comprises a second antibody that binds in a location that is compatible with MG97 binding. In some embodiments, the second antibody is A80-122P.

### Methods of Detecting Liver Damage

In some embodiments, the present disclosure provides a method for detecting liver damage or an increased risk of liver damage in a patient with alpha-1 antitrypsin deficiency (AATD), wherein the method comprises measuring the concentration of circulating polymerized alpha-1 antitrypsin (AAT) in the patient.

In some embodiments, the patient has a Z mutation (E342K), an S mutation (E264V), a Siiyama mutation (S53F), a Brescia mutation (G225R), an Mₘₐₗₜₒₙ (F52del) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115S) mutation, an X (E204K) mutation, a P_{lowell} (D256V) mutation, or a V333M mutation in the AAT protein.

In some embodiments, the patient has a Z mutation, an S mutation, a Siiyama mutation, a Brescia mutation, an Mₘₐₗₜₒₙ (F52de1) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), PiS+S14F mutations, an I50N mutation, an A58D mutation, an F227C mutation, a T249A mutation, or an H334D mutation in the AAT protein.

In some embodiments, the patient has a Z mutation in the AAT protein. In some embodiments, the patient is heterozygous for the Z mutation. In some embodiments, wherein the patient is heterozygous for the Z mutation, and has an additional AAT mutation associated with AATD. In some embodiments, the additional mutation associated with AATD is an S mutation (E264V;), a Siiyama mutation (S53F), a Brescia mutation (G225R), an Mₘₐₗₜₒₙ (F52del) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115S) mutation, an X (E204K) mutation, a P_{lowell} (D256V) mutation, or a V333M mutation in the AAT protein.

In some embodiments, the concentration of circulating polymerized AAT is determined by any of the methods disclosed herein.

In some embodiments, the patient is being treated with an AAT modulator or is being considered for treatment with an AAT modulator.

In some embodiments, the liver damage is cirrhosis, steatosis, liver inflammation (e.g., lobular and portal inflammation, and/or the presence of inflammatory foci), fibrosis, chronic hepatitis, portal hypertension, neonatal cholestasis, liver failure, or any combination thereof. In some embodiments, the liver damage is cirrhosis. In some embodiments, the liver damage is fibrosis. In some embodiments, the liver damage is liver inflammation. In some embodiments, the liver inflammation is lobular and/or portal inflammation. In some embodiments, the liver inflammation is the presence of inflammatory foci. In some embodiments, the liver damage is decompensated liver cirrhosis, end stage liver failure, hepatocellular carcinoma, or any combination thereof.

In some embodiments, elevated concentrations of circulating polymeric AAT are correlated with blood chemistry changes in circulating biomarkers of inflammation and liver health, e.g., ALT/AST. In some embodiments, the method of detecting liver damage or an increased risk of liver damage comprises measuring the concentration of circulating polymeric AAT and the concentration of one or more biomarker(s) of liver inflammation and/or liver health. In some embodiments, the one or more biomarker(s) of liver inflammation and/or liver health is ALT, AST, APRI (Aspartate aminotransferase-to-platelet ratio index), Fibrosis-4 Index, HepaScore, CRP, or any combination thereof. In some embodiments, the method comprises measuring the concentration of circulating polymeric AAT and measuring liver fibrosis by transient elastography (e.g., using a fibroscan).

In some embodiments, the detection of elevated levels of circulating polymeric AAT as compared to a healthy patient is indicative of liver damage, or an increased risk of liver damage. In some embodiments, the detection of any amount of circulating polymeric AAT is indicative of liver damage, or an increased risk of liver damage. In some embodiments, a plasma or serum concentration of about 1 μg/mL or higher of polymeric AAT is indicative of liver damage, or an increased risk of liver damage. In some embodiments, a plasma or serum concentration of about 2 μg/mL or higher of polymeric AAT is indicative of liver damage, or an increased risk of liver damage. In some embodiments, a plasma or serum concentration of about 3 μg/mL or higher of polymeric AAT is indicative of liver damage, or an increased risk of liver damage. In some embodiments, a plasma or serum concentration of about 4 μg/mL or higher of polymeric AAT is indicative of liver damage, or an increased risk of liver damage. In some embodiments, a plasma or serum concentration of about 5 μg/mL or higher of polymeric AAT is indicative of liver damage, or an increased risk of liver damage. In some embodiments, a plasma or serum concentration of about 7 μg/mL or higher of polymeric AAT is indicative of liver damage, or an increased risk of liver damage. In some embodiments, a plasma or serum concentration of about 10 μg/mL or higher of polymeric AAT is indicative of liver damage, or an increased risk of liver damage. In some embodiments, a plasma or serum concentration of about 15 μg/mL or higher of polymeric AAT is indicative of liver damage, or an increased risk of liver damage. In some embodiments, a plasma or serum concentration of about 20 μg/mL or higher of polymeric AAT is indicative of liver damage, or an increased risk of liver damage. In some embodiments, a plasma or serum concentration of about 25 μg/mL or higher of polymeric AAT is indicative of liver damage, or an increased risk of liver damage. In some embodiments, a plasma or serum concentration of about 30 μg/mL or higher of polymeric AAT is indicative of liver damage, or an increased risk of liver damage. In some embodiments, a plasma or serum concentration of about 40 μg/mL or higher of polymeric AAT is indicative of liver damage, or an increased risk of liver damage.

In some embodiments, a plasma or serum concentration of about 2 μg/mL to about 300 μg/mL is indicative of liver damage, or an increased risk of liver damage. In some embodiments, a plasma or serum concentration of about 2 μg/mL to about 250 μg/mL is indicative of liver damage, or an increased risk of liver damage. In some embodiments, a plasma or serum concentration of about 2 μg/mL to about 200 μg/mL is indicative of liver damage, or an increased risk of liver damage. In some embodiments, a plasma or serum concentration of about 2 μg/mL to about 150 μg/mL is indicative of liver damage, or an increased risk of liver damage. In some embodiments, a plasma or serum concentration of about 2 μg/mL to about 100 μg/mL is indicative of liver damage, or an increased risk of liver damage. In some embodiments, a plasma or serum concentration of about 2 μg/mL to about 60 μg/mL is indicative of liver damage, or an increased risk of liver damage.

The methods disclosed herein are the first biomarker-based assays that use circulating AAT polymer concentrations to monitor liver disease in AATD patients. Biomarker assays provide many significant advantages as compared to current methods for monitoring liver damage in AATD patients (i.e., liver biopsies and imaging methods). Liver biopsies are invasive, time consuming, and expensive. Imaging methods are also time consuming and expensive, and exhibit poor accuracy, as they show poor correlation with liver biopsy data, the current gold standard method for detecting liver damage in AATD patients. Moreover, imaging methods are also incapable of directly detecting AAT polymer, meaning that imaging techniques cannot distinguish AATD associated liver damage from damage caused by other factors (e.g., alcoholism). The methods disclosed herein are also more quantitative than liver biopsies, offer a broader dynamic range than liver biopsies, and are more sensitive than both biopsies and imaging techniques.

### Methods of Detecting an Increased Risk of a Liver-Related Clinical Outcome

In some embodiments, the present disclosure provides a method for detecting an increased risk of a liver-related clinical outcome in a patient with alpha-1 antitrypsin deficiency (AATD), wherein the method comprises measuring the concentration of circulating polymerized alpha-1 antitrypsin (AAT) in the patient.

In some embodiments, the patient has a Z mutation (E342K), an S mutation (E264V), a Siiyama mutation (S53F), a Brescia mutation (G225R), an Mₘₐₗₜₒₙ (F52del) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115 S) mutation, an X (E204K) mutation, a P_{lowell} (D256V) mutation, or a V333M mutation in the AAT protein.

In some embodiments, the patient has a Z mutation, an S mutation, a Siiyama mutation, a Brescia mutation, an Mₘₐₗₜₒₙ (F52de1) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), PiS+S14F mutations, an I50N mutation, an A58D mutation, an F227C mutation, a T249A mutation, or an H334D mutation in the AAT protein.

In some embodiments, the patient has a Z mutation in the AAT protein. In some embodiments, the patient is heterozygous for the Z mutation. In some embodiments, wherein the patient is heterozygous for the Z mutation, and has an additional AAT mutation associated with AATD. In some embodiments, the additional mutation associated with AATD is an S mutation (E264V), a Siiyama mutation (S53F), a Brescia mutation (G225R), an Mₘₐₗₜₒₙ (F52del) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115S) mutation, an X (E204K) mutation, a P_{lowell} (D256V) mutation, or a V333M mutation in the AAT protein.

In some embodiments, the concentration of circulating polymerized AAT is determined by any of the methods disclosed herein.

In some embodiments, the patient is being treated with an AAT modulator or is being considered for treatment with an AAT modulator.

In some embodiments, the liver-related clinical outcome is chosen from liver-related hospitalization, liver transplantation, and mortality. In some embodiments, the liver-related clinical outcome is liver-related hospitalization. In some embodiments, the liver-related clinical outcome is liver transplantation. In some embodiments, the liver-related clinical outcome is mortality.

In some embodiments, elevated concentrations of circulating polymeric AAT are correlated with blood chemistry changes in circulating biomarkers of inflammation and liver health, e.g., ALT/AST. In some embodiments, the method of detecting an increased risk of a liver-related clinical outcome comprises measuring the concentration of circulating polymeric AAT and the concentration of one or more biomarker(s) of liver inflammation and/or liver health. In some embodiments, the one or more biomarker(s) of liver inflammation and/or liver health is ALT, AST, APRI (Aspartate aminotransferase-to-platelet ratio index), Fibrosis-4 Index, HepaScore, CRP, or any combination thereof. In some embodiments, the method comprises measuring the concentration of circulating polymeric AAT and measuring liver fibrosis by transient elastography (e.g., using a fibroscan).

In some embodiments, the detection of elevated levels of circulating polymeric AAT as compared to a healthy patient is indicative of an increased risk of a liver-related clinical outcome. In some embodiments, the detection of any amount of circulating polymeric AAT is indicative of an increased risk of a liver-related clinical outcome. In some embodiments, a plasma or serum concentration of about 1 μg/mL or higher of polymeric AAT is indicative of an increased risk of a liver-related clinical outcome. In some embodiments, a plasma or serum concentration of about 2 μg/mL or higher of polymeric AAT is indicative of an increased risk of a liver-related clinical outcome. In some embodiments, a plasma or serum concentration of about 3 μg/mL or higher of polymeric AAT is indicative of an increased risk of a liver-related clinical outcome. In some embodiments, a plasma or serum concentration of about 4 μg/mL or higher of polymeric AAT is indicative of an increased risk of a liver-related clinical outcome. In some embodiments, a plasma or serum concentration of about 5 μg/mL or higher of polymeric AAT is indicative of an increased risk of a liver-related clinical outcome. In some embodiments, a plasma or serum concentration of about 7 μg/mL or higher of polymeric AAT is indicative of an increased risk of a liver-related clinical outcome. In some embodiments, a plasma or serum concentration of about 10 μg/mL or higher of polymeric AAT is indicative of an increased risk of a liver-related clinical outcome. In some embodiments, a plasma or serum concentration of about 15 μg/mL or higher of polymeric AAT is indicative of an increased risk of a liver-related clinical outcome. In some embodiments, a plasma or serum concentration of about 20 μg/mL or higher of polymeric AAT is indicative of an increased risk of a liver-related clinical outcome. In some embodiments, a plasma or serum concentration of about 25 μg/mL or higher of polymeric AAT is indicative of an increased risk of a liver-related clinical outcome. In some embodiments, a plasma or serum concentration of about 30 μg/mL or higher of polymeric AAT is indicative of an increased risk of a liver-related clinical outcome. In some embodiments, a plasma or serum concentration of about 40 μg/mL or higher of polymeric AAT is indicative of an increased risk of a liver-related clinical outcome.

In some embodiments, a plasma or serum concentration of about 2 μg/mL to about 300 μg/mL is indicative of an increased risk of a liver-related clinical outcome. In some embodiments, a plasma or serum concentration of about 2 μg/mL to about 250 μg/mL is indicative of an increased risk of a liver-related clinical outcome. In some embodiments, a plasma or serum concentration of about 2 μg/mL to about 200 μg/mL is indicative of an increased risk of a liver-related clinical outcome. In some embodiments, a plasma or serum concentration of about 2 μg/mL to about 150 μg/mL is indicative of an increased risk of a liver-related clinical outcome. In some embodiments, a plasma or serum concentration of about 2 μg/mL to about 100 μg/mL is indicative of an increased risk of a liver-related clinical outcome. In some embodiments, a plasma or serum concentration of about 2 μg/mL to about 60 μg/mL is indicative of an increased risk of a liver-related clinical outcome.

### Methods of Detecting Polymeric AAT in the Liver

In some embodiments, the present disclosure provides a method for detecting polymeric AAT in the liver of a patient with alpha-1 antitrypsin deficiency (AATD), wherein the method comprises measuring the concentration of circulating polymerized alpha-1 antitrypsin (AAT) in the patient.

In some embodiments, the patient has a Z mutation (E342K), an S mutation (E264 Siiyama mutation (S53F), a Brescia mutation (G225R), an Mₘₐₗₜₒₙ (F52del) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115 S) mutation, an X (E204K) mutation, a P_{lowell} (D256V) mutation, or a V333M mutation in the AAT protein.

In some embodiments, the patient has a Z mutation, an S mutation, a Siiyama mutation, a Brescia mutation, an Mₘₐₗₜₒₙ (F52de1) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), PiS+S14F mutations, an I50N mutation, an A58D mutation, an F227C mutation, a T249A mutation, or an H334D mutation in the AAT protein.

In some embodiments, the patient has a Z mutation in the AAT protein. In some embodiments, the patient is heterozygous for the Z mutation. In some embodiments, the patient is heterozygous for the Z mutation and has an additional AAT mutation associated with AATD. In some embodiments, the additional mutation associated with AATD is an S mutation (E264V), a Siiyama mutation (S53F), a Brescia mutation (G225R), an Mₘₐₗₜₒₙ (F52del) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115 S) mutation, an X (E204K) mutation, a P_{lowell} (D256V) mutation, or a V333M mutation in the AAT protein.

In some embodiments, the concentration of circulating polymerized AAT is measured using any of the methods disclosed herein.

In some embodiments, the patient is being treated with an AAT modulator or is being considered for treatment with an AAT modulator.

In some embodiments, the detection of elevated levels of circulating polymeric AAT as compared to a healthy patient indicates the presence of polymeric AAT in the patient's liver. In some embodiments, the detection of any amount of circulating polymeric AAT is indicative of liver damage, or an increased risk of liver damage. In some embodiments, a plasma or serum concentration of about 1 μg/mL or higher of polymeric AAT indicates the presence of polymeric AAT in the patient's liver. In some embodiments, a plasma or serum concentration of about 2 μg/mL or higher of polymeric AAT indicates the presence of polymeric AAT in the patient's liver. In some embodiments, a plasma or serum concentration of about 3 μg/mL or higher of polymeric AAT indicates the presence of polymeric AAT in the patient's liver. In some embodiments, a plasma or serum concentration of about 4 μg/mL or higher of polymeric AAT indicates the presence of polymeric AAT in the patient's liver. In some embodiments, a plasma or serum concentration of about 5 μg/mL or higher of polymeric AAT indicates the presence of polymeric AAT in the patient's liver. In some embodiments, a plasma or serum concentration of about 7 μg/mL or higher of polymeric AAT indicates the presence of polymeric AAT in the patient's liver. In some embodiments, a plasma or serum concentration of about 10 μg/mL or higher of polymeric AAT indicates the presence of polymeric AAT in the patient's liver. In some embodiments, a plasma or serum concentration of about 15 μg/mL or higher of polymeric AAT indicates the presence of polymeric AAT in the patient's liver. In some embodiments, a plasma or serum concentration of about 20 μg/mL or higher of polymeric AAT indicates the presence of polymeric AAT in the patient's liver. In some embodiments, a plasma or serum concentration of about 25 μg/mL or higher of polymeric AAT indicates the presence of polymeric AAT in the patient's liver. In some embodiments, a plasma or serum concentration of about 30 μg/mL or higher of polymeric AAT indicates the presence of polymeric AAT in the patient's liver. In some embodiments, a plasma or serum concentration of about 40 μg/mL or higher of polymeric AAT indicates the presence of polymeric AAT in the patient's liver.

In some embodiments, a plasma or serum concentration of about 2 μg/mL to about 300 μg/mL is indicative of polymeric AAT in the patient's liver. In some embodiments, a plasma or serum concentration of about 2 μg/mL to about 250 μg/mL is indicative of polymeric AAT in the patient's liver. In some embodiments, a plasma or serum concentration of about 2 μg/mL to about 200 μg/mL is indicative of polymeric AAT in the patient's liver. In some embodiments, a plasma or serum concentration of about 2 μg/mL to about 150 μg/mL is indicative of polymeric AAT in the patient's liver. In some embodiments, a plasma or serum concentration of about 2 μg/mL to about 100 μg/mL is indicative of polymeric AAT in the patient's liver. In some embodiments, a plasma or serum concentration of about 2 μg/mL to about 60 μg/mL is indicative of polymeric AAT in the patient's liver.

The presence of polymeric AAT in the liver of an AATD patient is highly correlated with liver damage or risk of future liver damage. Currently, the only available method for detecting AAT polymers in the liver is immunohistostaining of liver samples obtained by biopsy. As discussed above, liver biopsies are invasive, time consuming, and expensive. The methods disclosed herein are less invasive, more cost-effective, more sensitive, more quantitative, and have a broader dynamic range than existing, liver-biopsy based methods for quantifying liver AAT polymer concentrations.

Methods of Identifying Patients with an Improved Likelihood of Responding to AAT Modulator Treatment

Also disclosed herein is a method of identifying alpha-1 antitrypsin deficiency (AATD) patients with an increased likelihood of responding to treatment with an AAT modulator, wherein the method comprises measuring the concentration of circulating polymerized alpha-1 antitrypsin (AAT) in the patient.

In some embodiments, the method comprises measuring the concentration of circulating polymeric AAT using any one of the methods disclosed herein.

In some embodiments, the patient has a Z mutation (E342K), an S mutation (E264V), a Siiyama mutation (S53F), a Brescia mutation (G225R), an Mₘₐₗₜₒₙ (F52del) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115S) mutation, an X (E204K) mutation, a P_{lowell} (D256V) mutation, or a V333M mutation in the AAT protein.

In some embodiments, the patient has a Z mutation, an S mutation, a Siiyama mutation, a Brescia mutation, an Mₘₐₗₜₒₙ (F52de1) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), PiS+S14F mutations, an I50N mutation, an A58D mutation, an F227C mutation, a T249A mutation, or an H334D mutation in the AAT protein.

In some embodiments, the patient has a Z mutation in the AAT protein. In some embodiments, the patient is heterozygous for the Z mutation. In some embodiments, the patient is heterozygous for the Z mutation, and has an additional AAT mutation associated with AATD. In some embodiments, the additional mutation associated with AATD is an S mutation (E264V), a Siiyama mutation (S53F), a Brescia mutation ((2251t), an Mₘₐₗₜₒₙ (F52del) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115S) mutation, an X (E204K) mutation, a P_{lowell} (D256V) mutation, or a V333M mutation in the AAT protein.

In some embodiments, the AAT modulator is

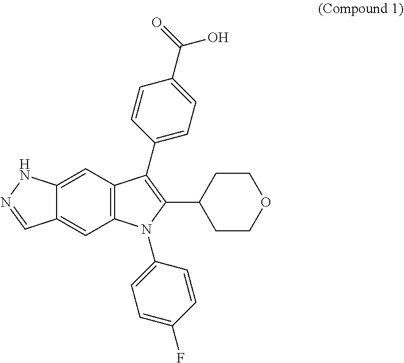 [SMILES: O=C(O)c1ccc(-c2c(C3CCOCC3)n(-c3ccc(F)cc3)c3cc4cn[nH]c4cc23)cc1]

or a pharmaceutically acceptable salt thereof.

In some embodiments, the AAT modulator is

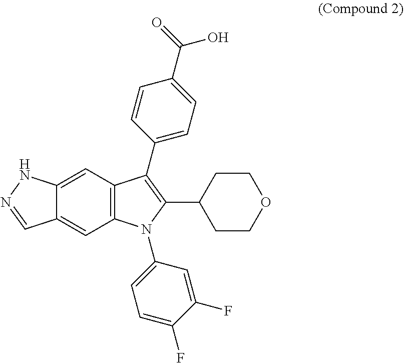 [SMILES: O=C(O)c1ccc(-c2c(C3CCOCC3)n(-c3ccc(F)c(F)c3)c3cc4cn[nH]c4cc23)cc1]

or a pharmaceutically acceptable salt thereof.

In some embodiments, the detection of elevated levels of circulating polymeric AAT as compared to a healthy patient is indicative of an increased likelihood of responding to treatment with an AAT modulator. In some embodiments, a plasma or serum concentration of about 1 μg/mL or higher of polymeric AAT is indicative of an increased likelihood of responding to treatment with an AAT modulator. In some embodiments, a plasma or serum concentration of about 2 μg/mL or higher of polymeric AAT is indicative of an increased likelihood of responding to treatment with an AAT modulator. In some embodiments, a plasma or serum concentration of about 3 μg/mL or higher of polymeric AAT is indicative of an increased likelihood of responding to treatment with an AAT modulator. In some embodiments, a plasma or serum concentration of about 4 μg/mL or higher of polymeric AAT is indicative of an increased likelihood of responding to treatment with an AAT modulator. In some embodiments, a plasma or serum concentration of about 5 μg/mL or higher of polymeric AAT is indicative of an increased likelihood of responding to treatment with an AAT modulator. In some embodiments, a plasma or serum concentration of about 7 μg/mL or higher of polymeric AAT is indicative of an increased likelihood of responding to treatment with an AAT modulator. In some embodiments, a plasma or serum concentration of about 10 μg/mL or higher of polymeric AAT is indicative of an increased likelihood of responding to treatment with an AAT modulator. In some embodiments, a plasma or serum concentration of about 15 μg/mL or higher of polymeric AAT is indicative of an increased likelihood of responding to treatment with an AAT modulator. In some embodiments, a plasma or serum concentration of about 20 μg/mL or higher of polymeric AAT is indicative of an increased likelihood of responding to treatment with an AAT modulator. In some embodiments, a plasma or serum concentration of about 25 μg/mL or higher of polymeric AAT is indicative of an increased likelihood of responding to treatment with an AAT modulator. In some embodiments, a plasma or serum concentration of about 30 μg/mL or higher of polymeric AAT is indicative of an increased likelihood of responding to treatment with an AAT modulator. In some embodiments, a plasma or serum concentration of about 40 μg/mL or higher of polymeric AAT is indicative of an increased likelihood of responding to treatment with an AAT modulator.

In some embodiments, a plasma or serum concentration of about 2 μg/mL to about 300 μg/mL is indicative of an increased likelihood of responding to treatment with an AAT modulator. In some embodiments, a plasma or serum concentration of about 2 μg/mL to about 250 μg/mL is indicative of an increased likelihood of responding to treatment with an AAT modulator. In some embodiments, a plasma or serum concentration of about 2 μg/mL to about 200 μg/mL is indicative of an increased likelihood of responding to treatment with an AAT modulator. In some embodiments, a plasma or serum concentration of about 2 μg/mL to about 150 μg/mL is indicative of an increased likelihood of responding to treatment with an AAT modulator. In some embodiments, a plasma or serum concentration of about 2 μg/mL to about 100 μg/mL is indicative of an increased likelihood of responding to treatment with an AAT modulator. In some embodiments, a plasma or serum concentration of about 2 μg/mL to about 60 μg/mL is indicative of an increased likelihood of responding to treatment with an AAT modulator.

Without being bound by theory, the methods disclosed herein are capable of identifying patients with an increased likelihood of responding to treatment with an AAT modulator in part because measuring circulating concentrations of AAT polymer allows patients whose liver damage is caused by factors other than AATD (e.g., patients having liver damage caused by alcoholism), and who are therefore unlikely to respond to treatment with an AAT modulator, to be filtered out.

### Methods for Treating AATD Patients

In some embodiments, the present disclosure provides a method of treating a patient with alpha-1 antitrypsin deficiency (AATD), wherein the method comprises: (i) measuring the concentration of circulating polymerized alpha-1 antitrypsin (AAT); and (ii) if the concentration of circulating AAT polymer is elevated as compared to a healthy patient, administering an AAT modulator, and if the concentration of AAT polymer is not elevated, not administering the AAT modulator.

In some embodiments, the concentration of circulating AAT polymer is measured using a method disclosed herein.

In some embodiments, the method comprises administering the AAT modulator if the plasma or serum concentration of AAT polymer is found to be about 1 μg/mL or higher, and not administering the AAT modulator if the concentration of AAT is found to be below 1 μg/mL. In some embodiments, the method comprises administering the AAT modulator if the plasma or serum concentration of AAT polymer is found to be about 2 μg/mL or higher, and not administering the AAT modulator if the concentration of AAT is found to be below 2 μg/mL. In some embodiments, the method comprises administering the AAT modulator if the plasma or serum concentration of AAT polymer is found to be about 3 μg/mL or higher, and not administering the AAT modulator if the concentration of AAT is found to be below 3 μg/mL. In some embodiments, the method comprises administering the AAT modulator if the plasma or serum concentration of AAT polymer is found to be about 4 μg/mL or higher, and not administering the AAT modulator if the concentration of AAT is found to be below 4 μg/mL. In some embodiments, the method comprises administering the AAT modulator if the plasma or serum concentration of AAT polymer is found to be about 5 μg/mL or higher, and not administering the AAT modulator if the concentration of AAT is found to be below 5 μg/mL. In some embodiments, the method comprises administering the AAT modulator if the plasma or serum concentration of AAT polymer is found to be about 7 μg/mL or higher, and not administering the AAT modulator if the concentration of AAT is found to be below 7 μg/mL. In some embodiments, the method comprises administering the AAT modulator if the plasma or serum concentration of AAT polymer is found to be about 10 μg/mL or higher, and not administering the AAT modulator if the concentration of AAT is found to be below 10 μg/mL. In some embodiments, the method comprises administering the AAT modulator if the plasma or serum concentration of AAT polymer is found to be about 15 μg/mL or higher, and not administering the AAT modulator if the concentration of AAT is found to be below 15 μg/mL. In some embodiments, the method comprises administering the AAT modulator if the plasma or serum concentration of AAT polymer is found to be about 20 μg/mL or higher, and not administering the AAT modulator if the concentration of AAT is found to be below 20 μg/mL. In some embodiments, the method comprises administering the AAT modulator if the plasma or serum concentration of AAT polymer is found to be about 25 μg/mL or higher, and not administering the AAT modulator if the concentration of AAT is found to be below 25 μg/mL. In some embodiments, the method comprises administering the AAT modulator if the plasma or serum concentration of AAT polymer is found to be about 30 μg/mL or higher, and not administering the AAT modulator if the concentration of AAT is found to be below 30 μg/mL. In some embodiments, the method comprises administering the AAT modulator if the plasma or serum concentration of AAT polymer is found to be about 40 μg/mL or higher, and not administering the AAT modulator if the concentration of AAT is found to be below 40 μg/mL.

In some embodiments, the method comprises administering the AAT modulator if the plasma or serum concentration of AAT polymer is found to be about 2 μg/mL to about 300 μg/mL. In some embodiments, the method comprises administering the AAT modulator if the plasma or serum concentration of AAT polymer is found to be about 2 μg/mL to about 250 μg/mL. In some embodiments, the method comprises administering the AAT modulator if the plasma or serum concentration of AAT polymer is found to be about 2 μg/mL to about 200 μg/mL. In some embodiments, the method comprises administering the AAT modulator if the plasma or serum concentration of AAT polymer is found to be about 2 μg/mL to about 150 μg/mL. In some embodiments, the method comprises administering the AAT modulator if the plasma or serum concentration of AAT polymer is found to be about 2 μg/mL to about 100 μg/mL. In some embodiments, the method comprises administering the AAT modulator if the plasma or serum concentration of AAT polymer is found to be about 2 μg/mL to about 60 μg/mL.

In some embodiments, the patient has a Z mutation (E342K), an S mutation (E264V), a Siiyama mutation (S53F), a Brescia mutation (G22510, an Mₘₐₗₜₒₙ (F52del) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115S) mutation, an X (E204K) mutation, a P_{lowell} (D256V) mutation, or a V333M mutation in the AAT protein.

In some embodiments, the patient has a Z mutation, an S mutation, a Siiyama mutation, a Brescia mutation, an Mₘₐₗₜₒₙ (F52de1) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), PiS+S14F mutations, an I50N mutation, an A58D mutation, an F227C mutation, a T249A mutation, or an H334D mutation in the AAT protein.

In some embodiments, the patient has a Z mutation in the AAT protein. In some embodiments, the patient is heterozygous for the Z mutation. In some embodiments, the patient is heterozygous for the Z mutation and has an additional AAT mutation associated with AATD. In some embodiments, the additional mutation associated with AATD is an S mutation (E264V), a Siiyama mutation (S531 4), a Brescia mutation (G225R), an Mₘₐₗₜₒₙ (F52del) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115S) mutation, an X (E204K) mutation, a P_{lowell} (D256V) mutation, or a V333M mutation in the AAT protein.

In some embodiments, the AAT modulator is

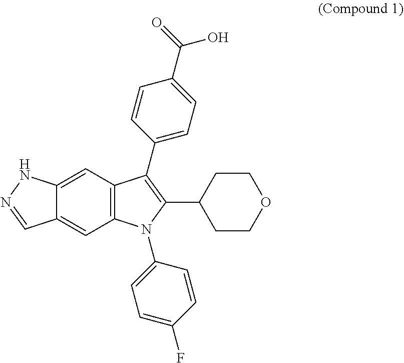 [SMILES: O=C(O)c1ccc(-c2c(C3CCOCC3)n(-c3ccc(F)cc3)c3cc4cn[nH]c4cc23)cc1]

or a pharmaceutically acceptable salt thereof.

In some embodiments, the AAT modulator is

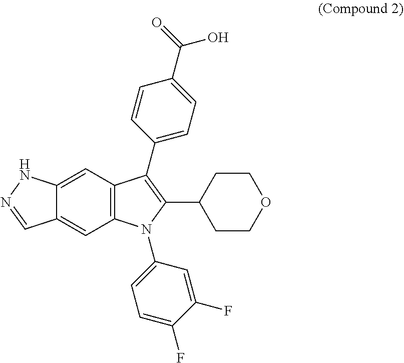 [SMILES: O=C(O)c1ccc(-c2c(C3CCOCC3)n(-c3ccc(F)c(F)c3)c3cc4cn[nH]c4cc23)cc1]

or a pharmaceutically acceptable salt thereof.

### Methods for Measuring the Efficacy of an AAT Modulator

Also disclosed herein is a method for measuring the efficacy of an alpha-1 antitrypsin (AAT) modulator, wherein the method comprises: (i) administering the AAT modulator to a patient; and (ii) determining the concentration of circulating polymerized AAT in the patient.

or or

In some embodiments, the method comprises determining the concentration of circulating polymerized AAT after administering the AAT modulator. In some embodiments, the method comprises determining the concentration of circulating polymerized AAT before and after administering the AAT modulator.

In some embodiments, the therapeutic efficacy of the AAT modulator is determined by measuring the change in the concentration of circulating polymerized AAT following administration of the AAT modulator.

In some embodiments, the concentration of AAT polymer is determined using a method disclosed herein.

In some embodiments, the AAT modulator is

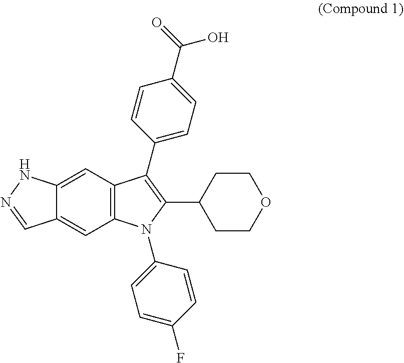 [SMILES: O=C(O)c1ccc(-c2c(C3CCOCC3)n(-c3ccc(F)cc3)c3cc4cn[nH]c4cc23)cc1]

or a pharmaceutically acceptable salt thereof.

In some embodiments, the AAT modulator is

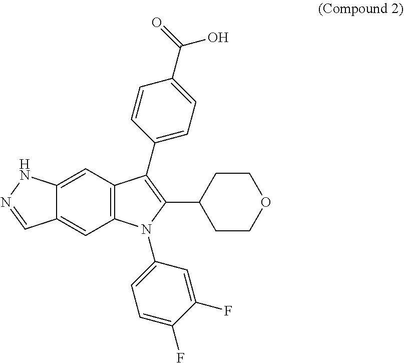 [SMILES: O=C(O)c1ccc(-c2c(C3CCOCC3)n(-c3ccc(F)c(F)c3)c3cc4cn[nH]c4cc23)cc1]

or a pharmaceutically acceptable salt thereof.

In some embodiments, the change in the concentration of circulating polymerized AAT is determined by obtaining a first measurement of circulating polymerized AAT concentrations shortly before beginning treatment with the AAT modulator, and obtaining a second measurement of circulating polymerized AAT concentrations shortly after beginning treatment with the AAT modulator. In some embodiments, the first measurement is obtained about three months or less before beginning treatment with the AAT modulator. In some embodiments, the first measurement is obtained about one month or less before beginning treatment with the AAT modulator. In some embodiments, the first measurement is obtained about one week or less before beginning treatment with the AAT modulator. In some embodiments, the second measurement is obtained one month or less after the first dose of the AAT modulator. In some embodiments, the second measurement is obtained two weeks or less after the first dose of the AAT modulator. In some embodiments, the second measurement is obtained one week or less after the first dose of the AAT modulator. In some embodiments, the second measurement is obtained 72 hours or less after the first dose of the AAT modulator.

Based on preclinical studies, AAT modulators with high activity are expected to rapidly decrease circulating polymerized AAT concentrations shortly after initial treatment. In some embodiments, an AAT modulator with high activity will decrease the concentration of circulating AAT polymer by approximately two-fold or more. In some embodiments, an AAT modulator with high activity will decrease the concentration of circulating AAT polymer by approximately three-fold or more. In some embodiments, an AAT modulator with high activity will decrease the concentration of circulating AAT polymer by approximately four-fold or more. In some embodiments, an AAT modulator with high activity will decrease the concentration of circulating AAT polymer by approximately five-fold or more. In some embodiments, an AAT modulator with high activity will decrease the concentration of circulating AAT polymer by approximately six-fold or more. In some embodiments, an AAT modulator with high activity will decrease the concentration of circulating AAT polymer by approximately seven-fold or more.

In some embodiments, an AAT modulator with high activity will decrease the concentration of circulating AAT polymer by approximately two- to ten-fold within a week of starting treatment. In some embodiments, an AAT modulator with high activity will decrease circulating AAT polymer concentrations by approximately three- to seven-fold within the first week of starting treatment.

### Methods for Determining an Efficacious Dosing Regimen for an AAT Modulator

In some embodiments, the present disclosure provides a method for determining an efficacious dosing regimen of an AAT modulator for treating alpha-1 antitrypsin deficiency (AATD) in a patient, wherein the method comprises measuring the concentration of circulating AAT polymer in the patient. In some embodiments, the method comprises measuring the concentration of circulating AAT polymer after administering the AAT modulator. In some embodiments, the method comprises measuring the concentration of circulating AAT polymer before and after administration of the AAT modulator.

In some embodiments, the method comprises increasing the dosage of the AAT modulator if the concentration of circulating polymerized AAT is found to be above about 10 μg/mL. In some embodiments, the method comprises increasing the dosage of the AAT modulator if the concentration of circulating polymerized AAT is found to be above about 5 μg/mL. In some embodiments, the method comprises increasing the dosage of the AAT modulator if the concentration of circulating polymerized AAT is found to be above about 2 μg/mL. In some embodiments, the method comprises increasing the dosage of the AAT modulator if the concentration of circulating polymerized AAT is found to be above about 1 μg/mL.

### Non-Limiting Example Embodiments

Without limitation, some embodiments of this disclosure include:

1. A method for measuring the concentration of polymerized alpha-1 antitrypsin (AAT) in a patient, wherein the method comprises:
i. using a first antibody to separate polymerized AAT from monomeric AAT in a patient sample, where the first antibody binds polymeric AAT with higher affinity than monomeric AAT;
ii. incubating the separated polymerized AAT with a second detection antibody, where the second antibody binds the AAT polymer; and
iii. measuring the amount of second antibody.

2. The method of Embodiment 1, wherein the sample is a BALF (bronchoalveolar lavage fluid), plasma, serum, sputum, urine, skin tissue, intestine, kidney, liver, lung, or blood sample.

3. The method of Embodiment 1, wherein the polymerized AAT is circulating polymerized AAT.

4. The method of any one of Embodiments 1-3, wherein the patient sample is a blood, serum, plasma, urine, or sputum sample.

5. The method of Embodiment 4, wherein the patient sample is a blood, serum, or plasma sample.

6. The method of Embodiment 5, wherein the patient sample is a blood sample.

7. The method of Embodiment 5, wherein the patient sample is a serum sample.

8. The method of Embodiment 5, wherein the patient sample is a plasma sample.

9. The method of any one of Embodiments 1-8, wherein the first antibody is 2C1 or ATZ11.

10. The method of Embodiment 9, wherein the first antibody is 2C1.

11. The method of Embodiment 9, wherein the first antibody is ATZ11.

12. The method of any one of Embodiments 1-11, wherein the second antibody binds in a location that is compatible with binding of 2C1.

13. The method of any one of Embodiments 1-11, wherein the second antibody binds in a location that is compatible with binding of ATZ11.

14. The method of any one of Embodiments 1-11, wherein the second antibody is A80-122P.

15. The method of any one of Embodiments 1-14, wherein the patient has alpha-1 antitrypsin deficiency (AATD).

16. The method of Embodiment 15, wherein the patient has a Z mutation, an S mutation, a Siiyama mutation, a Brescia mutation, a Z mutation (E342K), an S mutation (E264V), a Siiyama mutation (S53F), a Brescia mutation (G225R), an Mₘₐₗₜₒₙ (F52del) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115S) mutation, an X (E204K) mutation, a P_{lowell} (D256V) mutation, or a

V333M mutation in the AAT protein.

17. The method of Embodiment 15, wherein the patient has a Z mutation, an S mutation, a Siiyama mutation, a Brescia mutation, an Mₘₐₗₜₒₙ (F52del) mutation, a M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PiS+S14F mutation, an I50N mutation, an A58D mutation, an F227C mutation, a T249A mutation, or a H334D mutation in the AAT protein.

18. The method of Embodiment 15, wherein the patient has a Z mutation in the AAT protein.

19. The method of Embodiment 18, wherein the patient is heterozygous for the Z mutation.

20. The method of Embodiment 18, wherein the patient is heterozygous for the Z mutation, and has an additional AAT mutation associated with AATD.

21. The method of Embodiment 20, wherein the additional mutation associated with AATD is an S mutation (E264V), a Siiyama mutation (S53F), a Brescia mutation (G225R), an Mₘₐₗₜₒₙ (F52de1) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115S) mutation, an X (E204K) mutation, a P_{lowell} (D256V) mutation, or a V333M mutation in the AAT protein.

22. The method of Embodiment 18, wherein the patient has a PiZZ genotype.

23. The method of any one of Embodiments 1-22, wherein the method is an immunoassay.

24. The method of Embodiment 23, wherein the immunoassay is a radioimmunoassay, a chemiluminescent immunoassay, or a fluorescent immunoassay.

25. The method of Embodiment 24, wherein the chemiluminescent immunoassay is an electrochemiluminescent immunoassay.

26. The method of any one of Embodiments 23-25, wherein the immunoassay is an ELISA or an immunoprecipitation assay.

27. The method of Embodiment 26, wherein the immunoassay is an ELISA.

28. The method of any one of Embodiments 23-27, wherein the immunoassay is calibrated using heat-treated M-AAT, wherein the M-AAT has been heat-treated for 4-18 hours.

29. The method of Embodiment 28, wherein the M-AAT has been heat-treated for 4-12 hours.

30. The method of Embodiment 29, wherein the M-AAT has been heat-treated for 4-6 hours.

31. The method of any one of Embodiments 28-30, wherein the M-AAT has been heat-treated at 50° C. to 70° C.

32. The method of Embodiment 31, wherein the M-AAT has been heat-treated at about 60° C.

33. A kit for measuring the concentration of circulating polymerized alpha-1 antitrypsin (AAT) in a patient, wherein the kit comprises a first antibody that binds polymerized AAT with a higher affinity than monomeric AAT, and a second antibody that binds both monomeric and polymeric AAT.

34. The kit of Embodiment 33, wherein the first antibody is 2C1 or ATZ11.

35. The kit of Embodiment 34, wherein the first antibody is 2C1.

36. The kit of Embodiment 34, wherein the first antibody is ATZ11.

37. The kit of any one of Embodiments 33-36, wherein the second antibody binds in a location that is compatible with the binding of 2C1.

38. The kit of any one of Embodiments 33-36, wherein the second antibody binds in a location that is compatible with the binding of ATZ11.

39. The method of any one of Embodiments 33-36, wherein the second antibody is A80-122P.

40. A method of detecting liver damage or an increased risk of liver damage in a patient with alpha-1 antitrypsin deficiency (AATD), wherein the method comprises measuring the concentration of circulating polymerized alpha-1 antitrypsin (AAT) in the patient.

41. The method of Embodiment 40, wherein liver damage or the increased risk of liver damage is detected if an elevated amount of polymerized AAT is detected as compared to a healthy patient.

42. The method of Embodiment 40 or 41, wherein liver damage or the increased risk of liver damage is detected if the measured concentration of polymerized AAT in the plasma or serum of the patient is 1 μg/mL or higher.

43. The method of any one of Embodiments 40-42, wherein liver damage or an increased risk of liver damage is detected if the measured concentration of polymerized AAT in the plasma or serum of the patient is 2 μg/mL or higher.

44. The method of any one of Embodiments 40-43, wherein liver damage or an increased risk of liver damage is detected if the measured concentration of polymerized AAT in the plasma or serum of the patient is 10 μg/mL or higher.

45. The method of any one of Embodiments 40-44, wherein the patient has a Z mutation (E342K), an S mutation (E264V), a Siiyama mutation (S53F), a Brescia mutation (G225R), an Mₘₐₗₜₒₙ (F52del) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115S) mutation, an X (E204K) mutation, a P_{lowell} (D256V) mutation, or a V333M mutation in the AAT protein.

46. The method of Embodiment 45, wherein the patient has a Z mutation, an S mutation, a Siiyama mutation, a Brescia mutation, a Mₘₐₗₜₒₙ (F52del) mutation, a M_{wurzburg} (P369S) mutation, a Mₚᵢₛₐ (K259I) mutation, an E_{Taurisano} (K368E) mutation, a Y_{orzinuovi} (P391H) mutation, a Trento mutation (E75V), a PiS+S14F mutation, an I50N mutation, an A58D mutation, an F227C mutation, a T249A mutation, or a H334D mutation in the AAT protein.

47. The method of any one of Embodiments 40-46, wherein the patient has a Z mutation in the AAT protein.

48. The method of Embodiment 47, wherein the patient is heterozygous for the Z mutation.

49. The method of Embodiment 48, wherein the patient is heterozygous for the Z mutation, and has an additional AAT mutation associated with AATD.

50. The method of Embodiment 49, wherein the additional AAT mutation associated with AATD is an S mutation, a Siiyama mutation, a Brescia mutation, a Mₘₐₗₜₒₙ (F52del) mutation, a M_{wurzburg} (P369S) mutation, a Mₚᵢₛₐ (K259I) mutation, an E_{Taurisano} (K368E) mutation, a Y_{orzinuovi} (P391H) mutation, a Trento mutation (E75V), a PiS+S14F mutation, an I50N mutation, an A58D mutation, an F227C mutation, a T249A mutation, an I mutation, an F mutation, or a H334D mutation in the AAT protein.

51. The method of Embodiment 47, wherein the patient has a PiZZ genotype.

52. The method of any one of Embodiments 40-51, wherein the liver damage is cirrhosis, steatosis, lobular/portal inflammation, fibrosis, chronic hepatitis, portal hypertension, neonatal cholestasis, liver failure, or any combination thereof.

53. The method of any one of Embodiments 40-52, wherein the concentration of circulating polymerized AAT is measured using the method of any one of Embodiments 1-32.

54. The method of any one of Embodiments 40-53, wherein the patient has been administered an AAT modulator or is being considered for treatment with an AAT modulator.

55. A method of detecting polymeric alpha-1 antitrypsin (AAT) in the liver of a patient with alpha-1 antitrypsin deficiency (AATD), wherein the method comprises measuring the concentration of circulating polymerized AAT in the patient.

56. The method of Embodiment 55, wherein polymeric AAT is detected in the liver if the concentration of circulating polymerized AAT is elevated as compared to a healthy patient.

57. The method of Embodiment 55 or 56, wherein polymeric AAT is detected in the liver if the measured concentration of polymerized AAT in the plasma or serum of the patient is 1 μg/mL or higher.

58. The method of any one of Embodiments 55-57, wherein polymeric AAT is detected in the liver if the measured concentration of polymerized AAT in the plasma or serum of the patient is 2 μg/mL or higher.

59. The method of any one of Embodiments 55-58, wherein polymeric AAT is detected in the liver if the measured concentration of polymerized AAT in the plasma or serum of the patient is 10 μg/mL or higher.

60. The method of any one of Embodiments 55-59, wherein the patient has a Z mutation (E342K), an S mutation (E264V), a Siiyama mutation (S53F), a Brescia mutation (G225R), an Mₘₐₗₜₒₙ (F52del) mutation, an M_{wurzburg} (P369S) mutation, an Mₚᵢₛₐ (K2591) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115S) mutation, an X (E204K) mutation, a P_{lowell} (D256V) mutation, or a V333M mutation in the AAT protein.

61. The method of Embodiment 60, wherein the patient has a Z mutation in the AAT protein.

62. The method of Embodiment 61, wherein the patient is heterozygous for the Z mutation in the AAT protein.

63. The method of Embodiment 62, wherein the patient is heterozygous for the Z mutation, and has an additional AAT mutation associated with AATD.

64. The method of Embodiment 63, wherein the additional AAT mutation is an S mutation (E264V), a Siiyama mutation (S53F), a Brescia mutation (G225R), an Mₘₐₗₜₒₙ (F52del) mutation, an M_{wurzburg} (P369S) mutation, an Mₚᵢₛₐ(K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport (G}115S) mutation, an X (E204K) mutation, a P_{lowell} (D256V) mutation, or a V333M mutation in the AAT protein.

65. The method of Embodiment 61, wherein the patient has a PiZZ genotype.

66. The method of any one of Embodiments 55-65, wherein the concentration of circulating polymerized AAT is measured using the method of any one of Embodiments 1-32.

67. The method of any one of Embodiments 55-66, wherein the patient has been administered an AAT modulator or is being considered for treatment with an AAT modulator.

68. A method of identifying a patient with alpha-1 antitrypsin deficiency (AATD) that has an increased likelihood of responding to treatment with an AAT modulator, wherein the method comprises measuring the concentration of circulating polymerized alpha-1 antitrypsin (AAT) in the patient.

69. The method of Embodiment 68, wherein the concentration of circulating polymerized AAT is measured using the method of any one of Embodiments 1-32.

70. The method of Embodiment 68 or 69, wherein the patient has a Z mutation in the AAT protein.

71. The method of Embodiment 70, wherein the patient is heterozygous for the Z mutation in the AAT protein.

72. The method of Embodiment 71, wherein the patient is heterozygous for the Z mutation and has an additional mutation associated with AATD.

73. The method of Embodiment 70, wherein the patient has a PiZZ genotype.

74. The method of any one of Embodiments 68-73, wherein the AAT modulator is

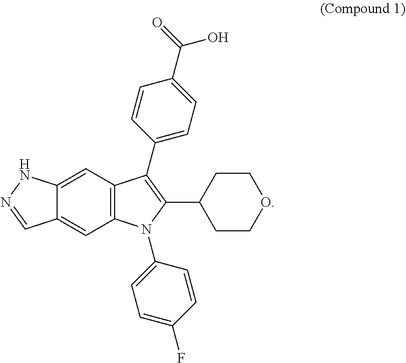 [SMILES: O=C(O)c1ccc(-c2c(C3CCOCC3)n(-c3ccc(F)cc3)c3cc4cn[nH]c4cc23)cc1]

75. The method of any one of Embodiments 68-74, wherein the patient with AATD is determined to have an increased likelihood of responding to treatment with an AAT modulator, if the concentration of circulating polymerized AAT is measured to be at least 1 μg/mL in a plasma or serum sample.

76. A method of treating a patient with alpha-1 antitrypsin deficiency (AATD), wherein the method comprises:
i. measuring the concentration of circulating polymerized alpha-1 antitrypsin (AAT); and
ii. if the concentration of circulating polymerized AAT is found to be at least 1 μg/mL, administering an AAT modulator, and if the concentration of circulating polymerized AAT is found to be less than 1 μg/mL, not administering an AAT modulator.

77. The method of Embodiment 76, wherein the concentration of circulating polymerized AAT is determined using the method of any one of Embodiments 1-32.

78. The method of Embodiment 76 or 77, wherein the patient has a Z mutation in the AAT protein.

79. The method of Embodiment 78, wherein the patient is heterozygous for the Z mutation.

80. The method of Embodiment 79, wherein the patient is heterozygous for the Z mutation, and has an additional mutation in AAT associated with AATD.

81. The method of Embodiment 78, wherein the patient has a PiZZ genotype.

82. The method of any one of Embodiments 76-81, wherein the AAT modulator is

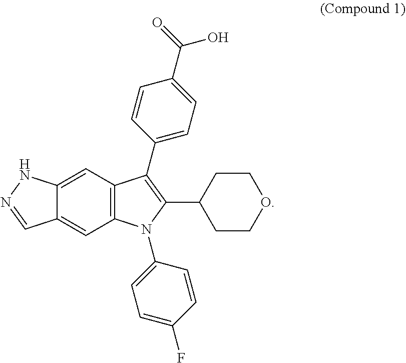 [SMILES: O=C(O)c1ccc(-c2c(C3CCOCC3)n(-c3ccc(F)cc3)c3cc4cn[nH]c4cc23)cc1]

83. A method for measuring the efficacy of an alpha-1 antitrypsin (AAT) modulator, wherein the method comprises:
i. administering the AAT modulator to a patient with alpha-1 antitrypsin deficiency (AATD); and
ii. measuring the change in the concentration of circulating polymerized AAT in the patient.

84. The method of Embodiment 83, wherein the change in the concentration of circulating polymerized AAT is determined after administering the AAT modulator.

85. The method of Embodiment 83 or 84, wherein the change in the concentration of circulating polymerized AAT is determined by obtaining a first measurement of the concentration of circulating polymerized AAT shortly before administering the AAT modulator, and obtaining a second measurement of the concentration of polymerized AAT shortly after administering the AAT modulator.

86. The method of Embodiment 85, wherein the first measurement is obtained within 3 months of administering the AAT modulator.

87. The method of Embodiment 85 or 86, wherein the second measurement is obtained within one month of administering the AAT modulator.

88. The method of any one of Embodiments 85-87, wherein the second measurement is obtained within one week of administering the AAT modulator.

89. The method of any one of Embodiments 83-88, wherein the concentration of circulating polymerized AAT is measured using the method of any one of Embodiments 1-32.

90. A method for determining an efficacious dosing regimen of an AAT modulator for treating alpha-1 antitrypsin deficiency (AATD) in a patient, wherein the method comprises measuring the change in the concentration of circulating polymerized AAT in the patient.

91. The method of Embodiment 90, wherein the method comprises measuring the change in the concentration of circulating polymerized AAT after administering the AAT modulator.

92. The method of Embodiment 90 or 91, wherein the method comprises measuring the concentration of circulating polymerized AAT before and after administering the AAT modulator.

93. The method of any one of Embodiments 90-92, wherein the dosage of the AAT modulator is increased if the concentration of circulating polymerized AAT is found to be above about 2 μg/mL.

94. The method of any one of Embodiments 90-93, wherein the dosage of the AAT modulator is increased if the concentration of circulating polymerized AAT is found to be above about 1 μg/mL.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### Example 1

### Characterization of AAT Polymer Reference Standards (Heat-Treated M-AAT)

### Generating Heat Polymerized Alpha-I Antitrypsin

Plasma purified alpha-1 antitrypsin (M-AAT) (EMD Millipore, 178251) was reconstituted at a nominal 1 mg/mL in ultrapure water and protein concentration determined by bicinchoninic acid assay (Pierce, 23225). M-AAT protein was aliquoted into 400 μL per 1.5 mL polypropylene microcentrifuge tube and heat treated at 60° C. for up to 24 hours while shaking (350 RPM) in a thermal heat block (Eppendorf, Thermomixer R) to induce polymerization. Polymerized M-AAT was stored at −70° C. prior to subsequent analysis and used as calibration standards in the polymer sandwich ELISA.

### Gel Electrophoresis

Polymerized M-AAT was resolved under non-denaturing conditions using 4 to 12% tris-glycine gels (Thermo Fisher Scientific, XP04125BOX) run at 220V for approximately 30 minutes with samples diluted in native sample buffer (Life Technologies, LC2673) and the equivalent of 0.5 μg (purified protein) or 1 μL(PiZZ human plasma) loaded per well. Gel proteins were transferred to a polyvinylidene difluoride membrane (Bio-Rad, 1704156) using the Trans-Blot® Turbo™ Transfer system (Bio-Rad). Membranes were rinsed with TBS-T (50 mM Tris-HCl, I50 mM NaCl with 0.1% Tween® 20), blocked for 1 hour at room temperature with TBS-T Block (TBS-T containing 5% (w/v) milk powder (Bio-Rad, 1706404)), and incubated with primary antibodies diluted in TBS-T Block overnight at 4° C. (while shaking). The primary antibodies used were goat anti-human AAT polyclonal HRP-conjugated antibody (Bethyl Laboratories, A80-122P) diluted to 0.5 μg/mL and mouse monoclonal anti-human AAT polymer antibody (Hycult Biotech, HM2289, clone 2C1) diluted to 0.1 μg/mL. Membranes were rinsed with TBS-T, and if necessary, incubated with secondary antibody goat anti-mouse IgG HRP-conjugated (Licor, 926-80010) diluted 1:10000 in TBS-T Block for 90 min at room temperature (while shaking). Membranes were rinsed with TBS-T, developed with enhanced chemiluminescence HRP substrate (Thermo Fisher Scientific, 34075), and visualized on a Licor imaging system (Licor, Odyssey® Fc).

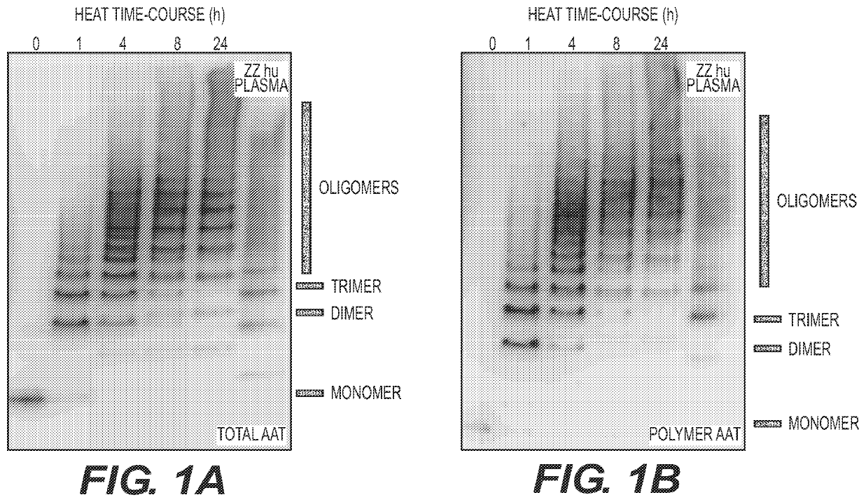

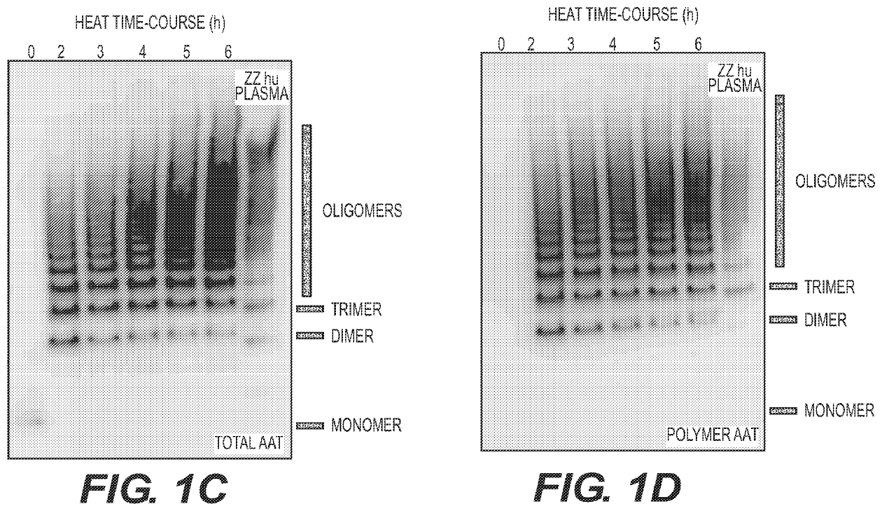

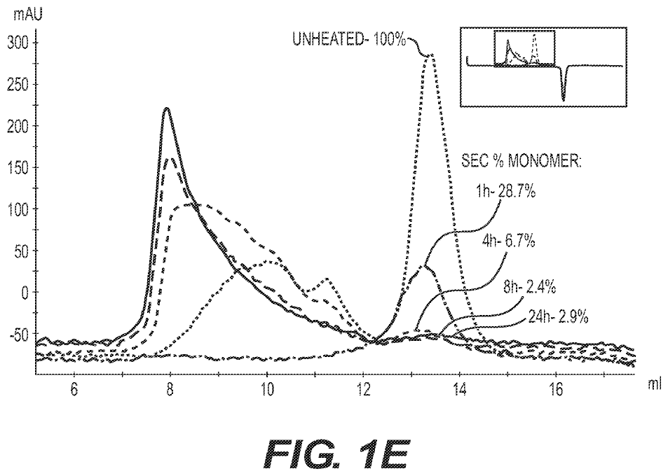

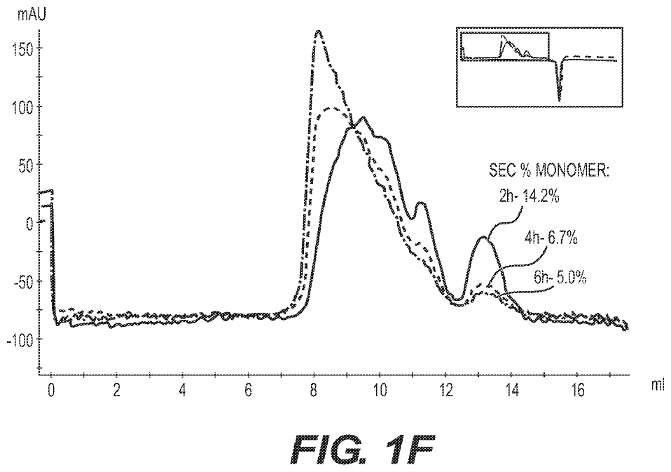

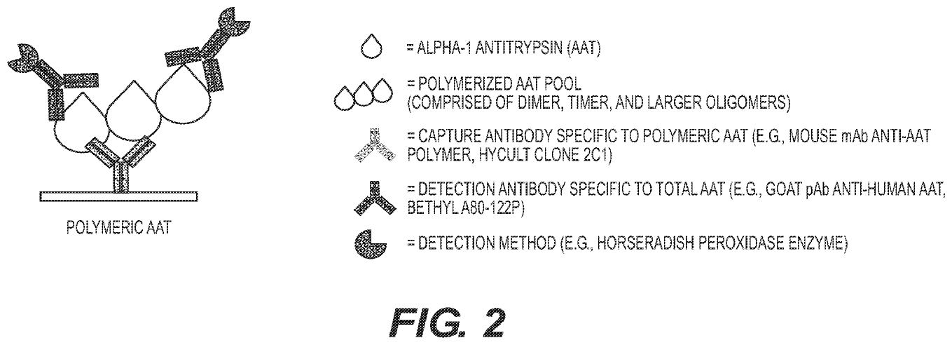

FIGS. 1A-1D depict Western blots of M-AAT samples heat-treated for 0-24 hours or 0-6 hours. The heat-treated samples comprise both AAT monomers and polymers, with longer heat treatment times producing a decrease in monomeric AAT and an increase in the number of larger molecular weight polymers. Heat-treating for 4-6 hours at 60° C. was found to produce an AAT polymer distribution similar to that found in AATD patients. Heat treatment for 1 hour produced AAT polymers that were smaller on average than those found in AATD patients, and heat-treatments of 8 hours and longer produced AAT polymers that were larger on average than those found in AATD patients.

### Size Exclusion Chromatography

The monomer:polymer ratio was determined using size exclusion chromatography (SEC). A Superdex 200 Increase 10/300 GL (Cytiva, 28-9909-44) was equilibrated in 20 mM HEPES pH 7.6 and 100 mM NaCl at 0.75 mL/min. Each sample was loaded separately onto the column, approximately 100 μL (1 mg/mL) per injection, and the elutions were monitored by measured absorbance at 205 and 280 nm. Post run, the 205 nm peaks were integrated using UNICORN software (Version 7.5).

SEC chromatograms of M-AAT polymer treated for 1-24 hours and 2-6 hours at 60° C. are shown in FIGS. 1E and 1F, respectively. AAT monomer was observed at ˜13.3 ml (A205). As in the Western blots of FIGS. 1A-1D, increasing heat treatment time was found to decrease the amount of monomer in the sample. After 6 hours of heat treatment, less than 5% of total protein is monomeric AAT.

### Example 2

### Detecting Circulating AAT Polymer in a Mouse Model Organism

A transgenic PiZ mouse model, on the genetic background of the C57BL/6 mouse strain with 8 copies of the human SERPINA1 gene with the Z mutation randomly integrated into the mouse genome, was utilized to detect circulating polymers in multiple tissues. Mouse urine and plasma were diluted in Assay Diluent to a final 25-fold and 500,000-fold dilutions, respectively. Bronchoalveolar lavage fluid (BALF) was collected by insertion of a canula into the trachea and flushing the lungs twice with the same 800 μL saline installation. The BALF was clarified by centrifugation, concentrated 10-fold by volume using a 30 kDa MW filter (Millipore, UFC503096), and stored at −70° C. prior to ELISA measurements. Samples were diluted 500-fold in Assay Diluent prior to measurement.

PiZ mouse tissues were harvested following whole-body perfusion with heparinized 0.9% saline (minimum 22 mL perfused over ˜1 min), snap frozen, and stored at −70° C. until lysis. Tissues were weighed and lysed in ice cold NP40 lysis buffer (Thermo Fisher Scientific, FNN0021) supplemented with a protease and phosphatase inhibitor cocktail (Thermo Fisher Scientific, 78440), 5 mM EDTA, and 1 mM phenylmethylsulfonyl fluoride (SelleckChem, S3025). Tissues were homogenized by mechanical disruption using metal beads (Omni International, 19-620) and a BeadRuptor12 instrument (Omni International) according to the following protocol (Speed=4, Time=10s, Cycles=4, Dwell Time=3s). Tissue lysate supernatants were separated from the insoluble fractions by centrifugation for 10 minutes at 10000 RPM using a benchtop centrifuge. Polymer in cell lysate supernatants was measured by ELISA and normalized by tissue mass.

### Polymer Sandwich ELISA

High bind EIA/RIA microplates (Costar, 3590) were coated with 100 μL mouse monoclonal anti-human AAT polymer antibody (Hycult Biotech, HM2289, clone 2C1) diluted to 1 μg/mL in 1× PBS (pH 7.4) and incubated for 90 minutes at 37° C. (while shaking) or alternatively overnight (up to 72 hours) at 4° C. (still). Plates were rinsed three times with 150 μL PBS-T (1XPBS with 0.05% (v/v) Tween® 20), blocked with 150 μL Assay Block (Meso Scale Discovery, R93AA-2), and incubated at room temperature for 90 minutes while shaking, then rinsed an additional three times with 150 μL PBS-T. Calibration standards (heat polymerized M-AAT) and samples were diluted in Assay Diluent (Assay Block diluted to 1% in 1×PBS), and 100 μL was loaded into each well and incubated overnight at 4° C. (still). Plates were rinsed three times with 150 μL PBS-T (washing away the unpolymerized, monomeric AAT before addition of a detection antibody against total AAT). 100 μL goat anti-human AAT polyclonal HRP-conjugated antibody (Bethyl Laboratories, A80-122P) diluted to 0.5 μg/mL in Assay Diluent was added to the plates, which were incubated at room temperature for approximately 2 hours while shaking. Plates were rinsed three times with 150 μL PBS-T, and 100 μL 3, 3′, 5, 5′-tetramethylbenzidine substrate (Sigma-Aldrich, T0440) was added per well and incubated at room temperature for 7-10 minutes while shaking. Following color development, 100 μL 1N sulfuric acid was added to stop the reaction and absorbance immediately measured at 450 nm with a microplate reader (Molecular Devices, SpectraMax M3). Calibration standards were fitted using a 4 PL equation in GraphPad Prism (Version 8.4.2).

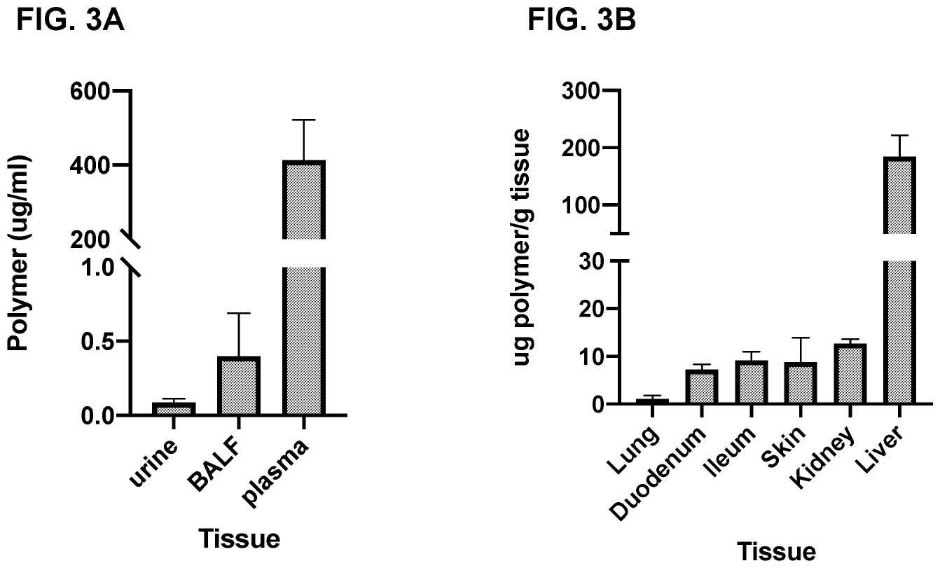

FIG. 3A shows AAT polymer concentrations measured in mouse urine, BALF, and plasma samples. FIG. 3B shows AAT polymer concentrations measured in various mouse tissue samples. For both FIGS. 3A and 3B, the data depicted are the mean and standard deviation from N=3 male PiZ hemizygous mice, approximately 12 weeks old.

### Example 3

### Detection Circulating AAT Polymers in Human Samples

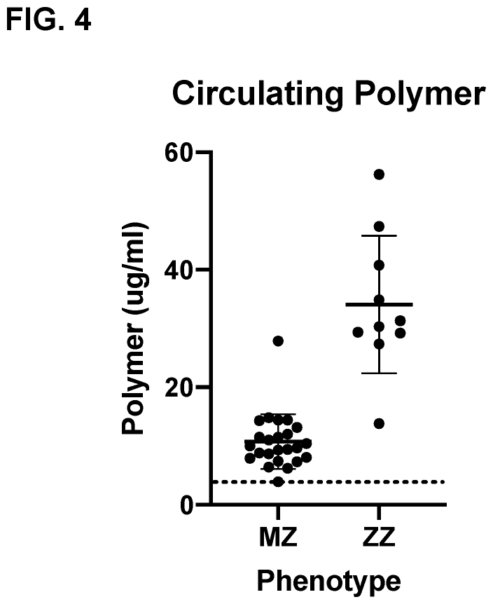

AAT polymer concentrations were measured by polymer sandwich ELISA in human serum and plasma samples collected and stored at −70° C. prior to analysis. Samples analyzed included plasma collected from 1 MZ and 2 ZZ patients and serum from all remaining donors. Samples were diluted 25,000- or 50,000-fold in Assay Diluent prior to measuring AAT polymer concentrations. AAT polymer concentrations were measured using the ELISA protocol of Example 2. Results are depicted in FIG. 4. 24/28 MZ donors and 10/10 ZZ donors showed detectable polymer.

### Example 4

### Optimized Sandwich ELISA for Detecting Circulating AAT Polymers in Human Samples

High bind EIA/RIA microplates (Costar, 3590) were coated with 100 μL mouse monoclonal anti-human AAT polymer antibody (Hycult Biotech, HM2289, clone 2C1) diluted to 2 μg/mL in carbonate-bicarbonate buffer (0.2M, pH 9.4) and incubated for 90 minutes at 37° C. (while shaking) or alternatively overnight (up to 72 hours) at 4° C. (still). Plates were rinsed three times with 300 μL PBS-T (1X PBS with 0.05% (v/v) Tween® 20), blocked with 150 μL Assay Diluent (Blocker™ Casein in PBS, Thermo Fisher Scientific, 37528), and incubated at room temperature for 90 minutes while shaking, then rinsed an additional three times with 300 PBS-T. Calibration standards (heat polymerized M-AAT) and samples were diluted in Assay Diluent, and 100 μL was loaded into each well and incubated overnight at 4° C. (still). Plates were rinsed four times with 300 μL PBS-T (washing away the unpolymerized, monomeric AAT before addition of a detection antibody against total AAT). 100 μL goat anti-human AAT polyclonal HRP-conjugated antibody (Bethyl Laboratories, A80-122P) diluted to 0.125 μg/mL in Assay Diluent was added to the plates and incubated at room temperature for 90 minutes while shaking. Plates were rinsed four times with 300 μL PBS-T, and 100 μL 3, 3′, 5, 5′-tetramethylbenzidine substrate (SeraCare, 5120-0047) was added per well and incubated at room temperature for 7-10 minutes while shaking. Following color development, 100 μL 1N sulfuric acid was added to stop the reaction and absorbance was immediately measured at 450 nm and 650 nm for background subtraction with a microplate reader (Molecular Devices, SpectraMax M3). Calibration standards were fitted using a 4 or 5 PL equation with 1/y2 weighting in GraphPad Prism (Version 8.4.2).

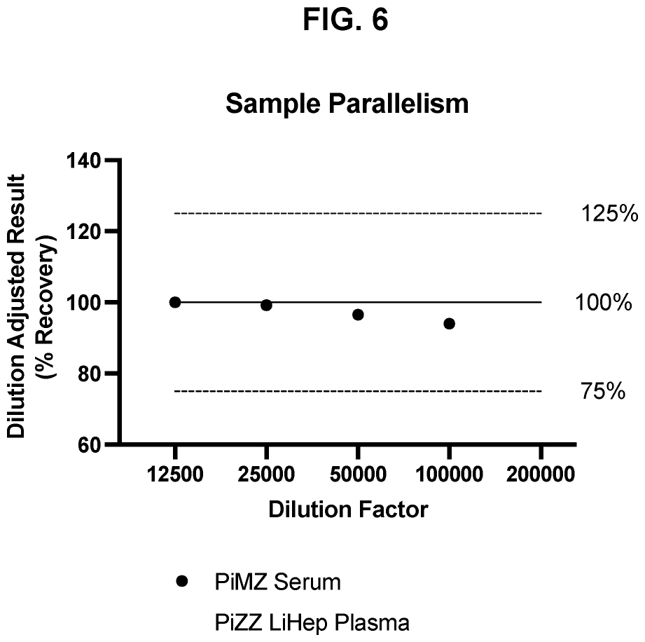

Parallelism was used to assess the concentration-response of AAT polymer to the calibration curve in individual donor MZ serum (Table 1) and ZZ plasma (Table 2) samples. Samples were analyzed in duplicate over a range of five two-fold serial dilutions starting at 1/12,500 to 1/200,000. Percent recovery was determined using the dilution adjusted concentration and compared back to the 1/12,500 result (FIG. 6) For each dilution, within the analytical assay range (50-25000 μg/mL), % Recovery was determined to be within 25.0% of the 1/12,500 result, where:

%Recovery=(observed result*dilution factor)/(dilution adjusted result at 1/12500)*100.

TABLE 1
PiMZ Serum Sample

| Dilution | Measured Result | Dilution Adjusted | % Recovery at |
|---|---|---|---|
| Factor | (pg/mL) | Result (μg/mL) | 12500 |
| 12500 | 526 | 6.58 | 100.0 |
| 25000 | 261 | 6.53 | 99.2 |
| 50000 | 127 | 6.35 | 96.6 |
| 100000 | 61.8 | 6.18 | 94.0 |
| 200000 | BLQ | | |

BLQ—below limit of quantification

TABLE 2
PiZZ Lithium Heparin Plasma Sample

| Dilution | Measured Result | Dilution Adjusted | % Recovery at |
|---|---|---|---|
| Factor | (pg/mL) | Result (μg/mL) | 12500 |
| 12500 | 1410 | 17.6 | 100.0 |
| 25000 | 714 | 17.9 | 101.3 |
| 50000 | 369 | 18.5 | 104.7 |
| 100000 | 193 | 19.3 | 109.5 |
| 200000 | 96.4 | 19.3 | 109.4 |

### Example 5

### Monitoring Pharmacodynamic Effect of Compound 2 Treatment to Lower Polymer Levels in a PiZ Mouse Model

Eleven week-old male PiZ mice, hemizygous for the Z variant of the human allele of AAT, were orally administered a suspension of either vehicle or Compound 2 QD at 50 and 100 mg/kg/day for 72 hours or 5, 16, 50, and 100 mg/kg/day for 28 days. At study termination, plasma was collected via cardiac puncture and liver tissue was collected and preserved in 10% neutral buffered formalin.

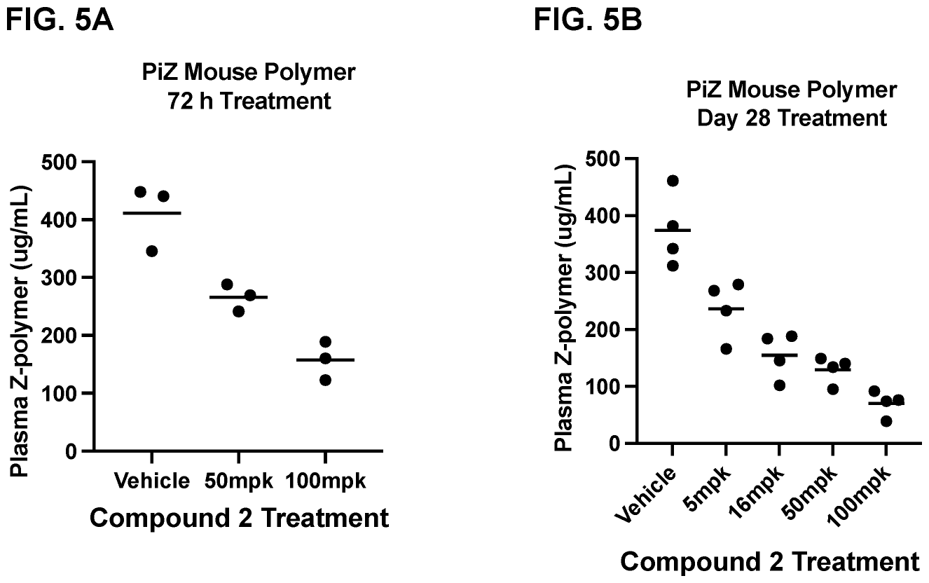

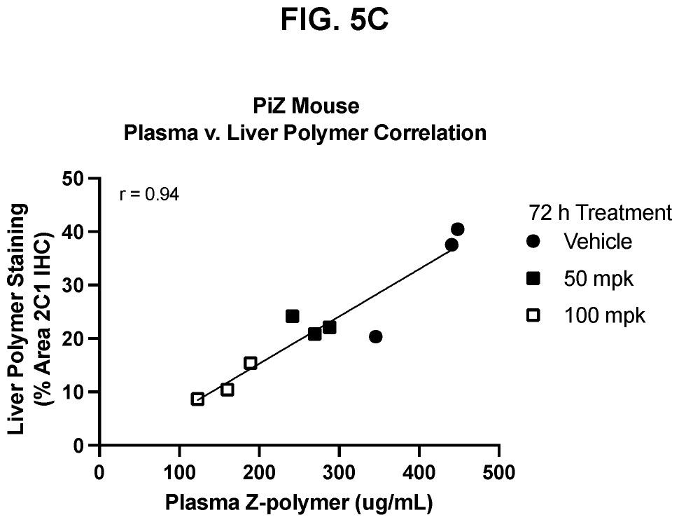

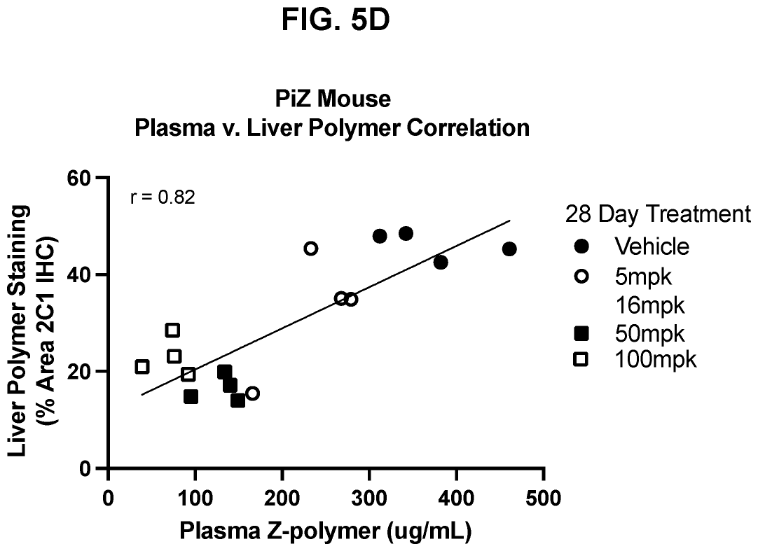

FIGS. 5A-5D show a pharmacodynamic dose response decrease in AAT plasma polymer levels in PiZ hemizygous mice after 72 hours (FIG. 5A) or 28 days (FIG. 5B) of treatment with vehicle or a small molecule AAT modulator, Compound 2, dosed QD at 50 and 100 mg/kg/day PO for 72 hours or 5, 16, 50, and 100 mg/kg/day PO for 28 days. Simple linear regression analysis demonstrated a strong correlation between levels of polymer in PiZ mouse plasma with liver polymer after (FIG. 5C) 72 hours, Pearson r=0.94 (95% CI: 0.75, 0.99), or (FIG. 5D) 28 days, Pearson r=0.82 (95% CI: 0.59-0.93), of AAT modulator treatment. Liver polymer staining was assessed as the % area of liver tissue that stains positively by monoclonal antibody 2C1 immunohistochemistry.

### Liver Histology and Image Analysis

Mouse liver tissue was paraffin embedded, and liver sections were prepared and stained as follows. 5-mm liver sections were stained for polymerized AAT with biotinylated mouse monoclonal (clone 2C1) antibody that recognizes polymerized AAT (Hycult Biotech Inc., Wayne, PA) using the following protocol. Briefly, slides were incubated with 2C1 primary antibody at 1:8000 dilution for 2 hours at room temperature, followed by detection with Discovery ChromoMap DAB Kit (Roche Diagnostics Corporation, Indianapolis, IN). The slides were counterstained with hematoxylin (Roche Diagnostics Corporation, Indianapolis, IN) and examined by light microscopy. All stains were performed using a Ventana automated platform. Liver polymer staining was determined as the % polymer 2C1 labeled area, including intracytoplasmic inclusion bodies and diffuse cytoplasmic labeling using HALO software version 3.2.1851 (Indica Labs, Albuquerque, NM).

### Polymer Sandwich ELISA

High bind EIA/RIA microplates (Costar, 3590) were coated with 100 μL mouse monoclonal anti-human AAT polymer antibody (Hycult Biotech, HM2289, clone 2C1) diluted to 2 μg/mL in carbonate-bicarbonate buffer (0.2M, pH 9.4) and incubated for 90 minutes at 37° C. (while shaking) or alternatively overnight (up to 72 hours) at 4° C. (still). Plates were rinsed three times with 300 μL PBS-T (1X PBS with 0.05% (v/v) Tween® 20), blocked with 150 μL Assay Diluent (Blocker™ Casein in PBS, Thermo Fisher Scientific, 37528), and incubated at room temperature for 90 minutes while shaking, then rinsed an additional three times with 300 μL PBS-T. Calibration standards and mouse plasma samples (diluted 1/50,000 and 1/500,000) were prepared in Assay Diluent and 100 μL was loaded into each well and incubated overnight at 4° C. (still). Plates were rinsed four times with 300 μL PBS-T, and 100 μL goat anti-human AAT polyclonal HRP-conjugated antibody (Bethyl Laboratories, A80-122P) diluted to 0.125 μg/mL in Assay Diluent added to the plates and incubated at room temperature for 90 minutes while shaking. Plates were rinsed four times with 300 μL PBS-T, and 100 μL 3, 3′, 5, 5′-tetramethylbenzidine substrate (SeraCare, 5120-0047) was added per well and incubated at room temperature for 7-10 minutes while shaking. Following color development, 100 μL 1N sulfuric acid was added to stop the reaction and absorbance was immediately measured at 450 nm and 650 nm for background subtraction with a microplate reader (Molecular Devices, SpectraMax M3). Calibration standards were fitted using a 5 PL equation with 1/y2 weighting in GraphPad Prism (Version 8.4.2).

### Example 6

### Specificity Testing of Circulating AAT Polymer Assay in the Prescence of Potentially Interfering Analytes

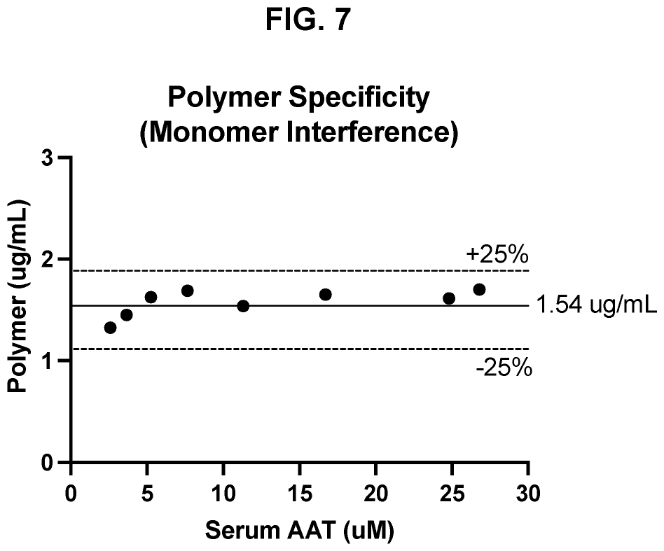

Specificity testing was performed to assess the ability of an assay of the present disclosure to detect AAT polymer in the presence of potentially interfering analytes (i.e., serum AAT assumed to be monomeric AAT in PiMM samples). Endogenous serum samples of different AATD phenotypes (PiZZ and PiMM) were mixed to create samples with a fixed level of AAT polymer (1.54 μg/mL dilution adjusted, or 123 pg/mL as measured in the polymer assay) and a range of serum AAT levels (2.59-26.8 μM). The percent recovery of polymer across the tested monomer range measured was 86.2% to 110.6%. There was no observed impact of AAT monomer interference in samples with up to 26.8 μM when the polymer concentration measured was 1.54 μg/mL (FIG. 7).

### Serum AAT

Serum AAT concentration was determined by immunonephelometric assay (Siemans 510(k) cleared A1AT kit) for the neat PiZZ and PiMM samples.

### Polymer Sandwich ELISA

High bind EIA/RIA microplates (Costar, 3590) were coated with 100 μL mouse monoclonal anti-human AAT polymer antibody (Hycult Biotech, HM2289, clone 2C1) diluted to 2 μg/mL in carbonate-bicarbonate buffer (0.2M, pH 9.4) and incubated for 90 minutes at 37° C. (while shaking) or alternatively overnight (up to 72 hours) at 4° C. (still). Plates were rinsed three times with 300 μL PBS-T (1X PBS with 0.05% (v/v) Tween® 20), blocked with 150 μL Assay Diluent (Blocker™ Casein in PBS, Thermo Fisher Scientific, 37528), and incubated at room temperature for 90 minutes while shaking, then rinsed an additional three times with 300 μL PBS-T. Calibration standards and samples were diluted in Assay Diluent and 100 μL was loaded into each well and incubated overnight at 4° C. (still). Plates were rinsed four times with 300 μL PBS-T, and 100 μL goat anti-human AAT polyclonal HRP-conjugated antibody (Bethyl Laboratories, A80-122P) diluted to 0.125 μg/mL in Assay Diluent was added to the plates, which were incubated at room temperature for 90 minutes while shaking. Plates were rinsed four times with 300 μL PBS-T, and 100 μL 3, 3′, 5, 5′ -tetramethylbenzidine substrate (SeraCare, 5120-0047) was added per well and incubated at room temperature for 7-10 minutes while shaking. Following color development, 100 μL 1N sulfuric acid was added to stop the reaction and absorbance was immediately measured at 450 nm and 650 nm for background subtraction with a microplate reader (Molecular Devices, SpectraMax, 384 plate reader). Calibration standards were fitted using a 5 PL equation with 1/y² weighting in Thermo Scientific Watson LIMS (v7.4.2 or higher).

### REFERENCES

Bergin, David A., et al. “The circulating proteinase inhibitor α-1 antitrypsin regulates neutrophil degranulation and autoimmunity.” Science Translational Medicine 6.217 (2014): 217ra1-217ra1.

Clark, Virginia C., et al. “Clinical and histologic features of adults with alpha-1 antitrypsin deficiency in a non-cirrhotic cohort.” Journal of Hepatology 69.6 (2018): 1357-1364.

Fra, Anna M., et al. “Three new alphal-antitrypsin deficiency variants help to define a C-terminal region regulating conformational change and polymerization.” PLoS One 7.6 (2012): e38405.

Geraghty, Patrick, et al. “α1-Antitrypsin activates protein phosphatase 2A to counter lung inflammatory responses.” American journal of respiratory and critical care medicine 190.11 (2014): 1229-1242.

Janciauskiene, Sabina, et al. “Detection of circulating and endothelial cell polymers of Z and wild type al-antitrypsin by a monoclonal antibody.” Journal of Biological Chemistry 277.29 (2002): 26540-26546.

Janciauskiene, Sabina, et al. “Differential detection of PAS-positive inclusions formed by the Z, Siiyama, and Mmalton variants of α1-antitrypsin.” Hepatology 40.5 (2004): 1203-1210.

Laffranchi, Mattia, et al. “Heteropolymerization of α-1-antitrypsin mutants in cell models mimicking heterozygosity.” Human Molecular Genetics 27.10 (2018): 1785-1793.

Matamala, Nerea, et al. “Characterization of novel missense variants of SERPINA1 gene causing Alpha-1 antitrypsin deficiency.” American journal of respiratory cell and molecular biology 58.6 (2018): 706-716.

Miranda, Elena, et al. “The pathological Trento variant of alpha-1-antitrypsin (E75V) shows nonclassical behaviour during polymerization.” The FEBS Journal 284.13 (2017): 2110-2126.

Patel, D. et al. “Alpha-1-Antitrypsin Deficiency Liver Disease.” Clinics in Liver Disease 22 (4 (2018): 643-655

Strnad, Pavel, et al. “Heterozygous carriage of the alphal -antitrypsin Pi* Z variant increases the risk to develop liver cirrhosis.” Gut 68.6 (2019): 1099-1107.

1. A method for measuring the concentration of polymerized alpha-1 antitrypsin (AAT) in a patient, wherein the method comprises:
i. using a first antibody to separate polymerized AAT from monomeric AAT in a patient sample, where the first antibody binds polymeric AAT with higher affinity than monomeric AAT;
ii. incubating the separated polymerized AAT with a second detection antibody, where the second antibody binds the AAT polymer; and
iii. measuring the amount of second antibody.

2. The method of claim 1, wherein the sample is a BALF (bronchoalveolar lavage fluid), plasma, serum, sputum, urine, skin tissue, intestine, kidney, liver, lung, or blood sample.

3. The method of claim 1, wherein the polymerized AAT is circulating polymerized AAT.

4. The method of any one of claims 1-3, wherein the patient sample is a blood, serum, plasma, urine, or sputum sample.

5. The method of claim 4, wherein the patient sample is a blood, serum, or plasma sample.

6. The method of claim 5, wherein the patient sample is a blood sample.

7. The method of claim 5, wherein the patient sample is a serum sample.

8. The method of claim 5, wherein the patient sample is a plasma sample.

9. The method of any one of claims 1-8, wherein the first antibody is 2C1 or ATZ11.

10. The method of claim 9, wherein the first antibody is 2C1.

11. The method of claim 9, wherein the first antibody is ATZ11.

12. The method of any one of claims 1-8, wherein the first antibody is LG96, MG97, or an antigen-binding fragment thereof.

13. The method of any one of claims 1-12, wherein the second antibody binds in a location that is compatible with binding of 2C1.

14. The method of any one of claims 1-12, wherein the second antibody binds in a location that is compatible with binding of ATZ11.

15. The method of any one of claims 1-12, wherein the second antibody is A80-122P.

16. The method of any one of claims 1-15, wherein the patient has alpha-1 antitrypsin deficiency (AATD).

17. The method of claim 16, wherein the patient has a Z mutation, an S mutation, a Siiyama mutation, a Brescia mutation, a Z mutation (E342K), an S mutation (E264V), a Siiyama mutation (S53F), a Brescia mutation (G225R), an Mₘₐₗₜₒₙ (F52del) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115S) mutation, an X (E204K) mutation, a P_{lowell} (D256V) mutation, or a V333M mutation in the AAT protein.

18. The method of claim 16, wherein the patient has a Z mutation, an S mutation, a Siiyama mutation, a Brescia mutation, an Mₘₐₗₜₒₙ (F52del) mutation, a M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PiS+S14F mutation, an I50N mutation, an A58D mutation, an F227C mutation, a T249A mutation, or a H334D mutation in the AAT protein.

19. The method of claim 16, wherein the patient has a Z mutation in the AAT protein.

20. The method of claim 19, wherein the patient is heterozygous for the Z mutation.

21. The method of claim 19, wherein the patient is heterozygous for the Z mutation, and has an additional AAT mutation associated with AATD.

22. The method of claim 21, wherein the additional mutation associated with AATD is an S mutation (E264V), a Siiyama mutation (S53F), a Brescia mutation (G225R), an Mₘₐₗₜₒₙ (F52del) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115S) mutation, an X (E204K) mutation, a P_{lowell} (D256V) mutation, or a V333M mutation in the AAT protein.

23. The method of claim 19, wherein the patient has a PiZZ genotype.

24. The method of any one of claims 1-23, wherein the method is an immunoassay.

25. The method of claim 24, wherein the immunoassay is a radioimmunoassay, a chemiluminescent immunoassay, or a fluorescent immunoassay.

26. The method of claim 25, wherein the chemiluminescent immunoassay is an electrochemiluminescent immunoassay.

27. The method of any one of claims 24-26, wherein the immunoassay is an ELISA or an immunoprecipitation assay.

28. The method of claim 27, wherein the immunoassay is an ELISA.

29. The method of any one of claims 24-28, wherein the immunoassay is calibrated using heat-treated M-AAT, wherein the M-AAT has been heat-treated for 4-18 hours.

30. The method of claim 29, wherein the M-AAT has been heat-treated for 4-12 hours.

31. The method of claim 30, wherein the M-AAT has been heat-treated for 4-6 hours.

32. The method of any one of claims 29-31, wherein the M-AAT has been heat-treated at 50° C. to 70° C.

33. The method of claim 32, wherein the M-AAT has been heat-treated at about 60° C.

34. A kit for measuring the concentration of circulating polymerized alpha-1 antitrypsin (AAT) in a patient, wherein the kit comprises a first antibody that binds polymerized AAT with a higher affinity than monomeric AAT, and a second antibody that binds both monomeric and polymeric AAT.

35. The kit of claim 34, wherein the first antibody is 2C1 or ATZ11.

36. The kit of claim 35, wherein the first antibody is 2C1.

37. The kit of claim 35, wherein the first antibody is ATZ11.

38. The kit of claim 34, wherein the first antibody is LG96, MG97, or an antigen-binding fragment thereof.

39. The kit of any one of claims 34-38, wherein the second antibody binds in a location that is compatible with the binding of 2C1.

40. The kit of any one of claims 34-38, wherein the second antibody binds in a location that is compatible with the binding of ATZ11.

41. The kit of any one of claims 34-38, wherein the second antibody binds in a location that is compatible with the binding of LG96, MG97, or an antigen-binding fragment thereof.

42. The method of any one of claims 34-38, wherein the second antibody is A80-122P.

43. A method of detecting liver damage, an increased risk of liver damage, or an increased risk of a liver-related clinical outcome in a patient with alpha-1 antitrypsin deficiency (AATD), wherein the method comprises measuring the concentration of circulating polymerized alpha-1 antitrypsin (AAT) in the patient.

44. The method of claim 43, wherein liver damage, the increased risk of liver damage, or the increased risk of a liver-related clinical outcome is detected if an elevated amount of polymerized AAT is detected as compared to a healthy patient.

45. The method of claim 43 or 44, wherein liver damage, the increased risk of liver damage, or the increased risk of a liver-related clinical outcome is detected if the measured concentration of polymerized AAT in the plasma or serum of the patient is 1 μg/mL or higher.

46. The method of any one of claims 43-45, wherein liver damage, the increased risk of liver damage, or the increased risk of a liver-related clinical outcome is detected if the measured concentration of polymerized AAT in the plasma or serum of the patient is 2 μg/mL or higher.

47. The method of any one of claims 43-46, wherein liver damage, the increased risk of liver damage, or the increased risk of a liver-related clinical outcome is detected if the measured concentration of polymerized AAT in the plasma or serum of the patient is 10 μg/mL or higher.

48. The method of any one of claims 43-47, wherein the patient has a Z mutation (E342K), an S mutation (E264V), a Siiyama mutation (S53F), a Brescia mutation (G225R), an Mₘₐₗₜₒₙ (F52del) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115S) mutation, an X (E204K) mutation, a P_{lowell} (D256V) mutation, or a V333M mutation in the AAT protein.

49. The method of claim 48, wherein the patient has a Z mutation, an S mutation, a Siiyama mutation, a Brescia mutation, a Mₘₐₗₜₒₙ (F52del) mutation, a M_{wurzburg} (P369S) mutation, a Mₚᵢₛₐ (K259I) mutation, an E_{Taurisano} (K368E) mutation, a Y_{orzinuovi} (P391H) mutation, a Trento mutation (E75V), a PiS+S14F mutation, an I50N mutation, an A58D mutation, an F227C mutation, a T249A mutation, or a H334D mutation in the AAT protein.

50. The method of any one of claims 43-49, wherein the patient has a Z mutation in the AAT protein.

51. The method of claim 50, wherein the patient is heterozygous for the Z mutation.

52. The method of claim 51, wherein the patient is heterozygous for the Z mutation, and has an additional AAT mutation associated with AATD.

53. The method of claim 52, wherein the additional AAT mutation associated with AATD is an S mutation, a Siiyama mutation, a Brescia mutation, a Mₘₐₗₜₒₙ (F52del) mutation, a M_{wurzburg} (P369S) mutation, a Mₚᵢₛₐ (K259I) mutation, an E_{Taurisano} (K368E) mutation, a Y_{orzinuovi} (P391H) mutation, a Trento mutation (E75V), a PiS+S14F mutation, an I50N mutation, an A58D mutation, an F227C mutation, a T249A mutation, an I mutation, an F mutation, or a H334D mutation in the AAT protein.

54. The method of claim 50, wherein the patient has a PiZZ genotype.

55. The method of any one of claims 43-54, wherein the liver damage is cirrhosis, steatosis, lobular/portal inflammation, fibrosis, chronic hepatitis, portal hypertension, neonatal cholestasis, liver failure, or any combination thereof.

56. The method of any one of claims 43-54, wherein the liver-related clinical outcome is chosen from liver-related hospitalization, liver transplantation, and mortality.

57. The method of any one of claims 43-56, wherein the concentration of circulating polymerized AAT is measured using the method of any one of claims 1-32.

58. The method of any one of claims 43-57, wherein the patient has been administered an AAT modulator or is being considered for treatment with an AAT modulator.

59. A method of detecting polymeric alpha-1 antitrypsin (AAT) in the liver of a patient with alpha-1 antitrypsin deficiency (AATD), wherein the method comprises measuring the concentration of circulating polymerized AAT in the patient.

60. The method of claim 59, wherein polymeric AAT is detected in the liver if the concentration of circulating polymerized AAT is elevated as compared to a healthy patient.

61. The method of claim 59 or 60, wherein polymeric AAT is detected in the liver if the measured concentration of polymerized AAT in the plasma or serum of the patient is 1 μg/mL or higher.

62. The method of any one of claims 59-61, wherein polymeric AAT is detected in the liver if the measured concentration of polymerized AAT in the plasma or serum of the patient is 2 μg/mL or higher.

63. The method of any one of claims 59-62, wherein polymeric AAT is detected in the liver if the measured concentration of polymerized AAT in the plasma or serum of the patient is 10 μg/mL or higher.

64. The method of any one of claims 59-63, wherein the patient has a Z mutation (E342K), an S mutation (E264V), a Siiyama mutation (S53F), a Brescia mutation (G225R), an Mₘₐₗₜₒₙ (F52de1) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115S) mutation, an X (E204K) mutation, a P_{lowell} (D256V) mutation, or a V333M mutation in the AAT protein.

65. The method of claim 64, wherein the patient has a Z mutation in the AAT protein.

66. The method of claim 65, wherein the patient is heterozygous for the Z mutation in the AAT protein.

67. The method of claim 66, wherein the patient is heterozygous for the Z mutation, and has an additional AAT mutation associated with AATD.

68. The method of claim 67, wherein the additional AAT mutation is an S mutation (E264V), a Siiyama mutation (S53F), a Brescia mutation (G225R), an Mₘₐₗₜₒₙ (F52del) mutation, an M_{wurzburg} (P369S) mutation, an Mₚᵢₛₐ (K259I) mutation, an E_{Taurisano} (K368E) mutation, a Y_{orzinuovi} (P391H) mutation, a Trento mutation (E75V), a PIS+S14F mutation, an I50N mutation, an A58D mutation, an F227C mutation, a T249A mutation, an I (R39C) mutation, an F (R223C) mutation, an H334D mutation, a V333M mutation, a Z_{bristol} (T85M) mutation, a Q0_{ludwigshafen} (I92N) mutation, a Q0_{newport} (G115S) mutation, an X (E204K) mutation, a P_{lowell} (D256V) mutation, or a V333M mutation in the AAT protein.

69. The method of claim 65, wherein the patient has a PiZZ genotype.

70. The method of any one of claims 59-69, wherein the concentration of circulating polymerized AAT is measured using the method of any one of claims 1-32.

71. The method of any one of claims 59-70, wherein the patient has been administered an AAT modulator or is being considered for treatment with an AAT modulator.

72. A method of identifying a patient with alpha-1 antitrypsin deficiency (AATD) that has an increased likelihood of responding to treatment with an AAT modulator, wherein the method comprises measuring the concentration of circulating polymerized alpha-1 antitrypsin (AAT) in the patient.

73. The method of claim 72, wherein the concentration of circulating polymerized AAT is measured using the method of any one of claims 1-32.

74. The method of claim 72 or 73, wherein the patient has a Z mutation in the AAT protein.

75. The method of claim 74, wherein the patient is heterozygous for the Z mutation in the AAT protein.

76. The method of claim 75, wherein the patient is heterozygous for the Z mutation and has an additional mutation associated with AATD.

77. The method of claim 74, wherein the patient has a PiZZ genotype.

78. The method of any one of claims 72-77, wherein the AAT modulator is

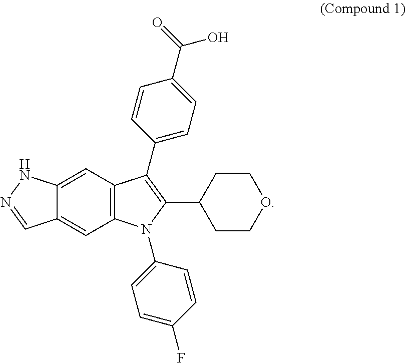 [SMILES: O=C(O)c1ccc(-c2c(C3CCOCC3)n(-c3ccc(F)cc3)c3cc4cn[nH]c4cc23)cc1]

79. The method of any one of claims 72-78, wherein the patient with AATD is determined to have an increased likelihood of responding to treatment with an AAT modulator, if the concentration of circulating polymerized AAT is measured to be at least 1 μg/mL in a plasma or serum sample.

80. A method of treating a patient with alpha-1 antitrypsin deficiency (AATD), wherein the method comprises:
i. measuring the concentration of circulating polymerized alpha-1 antitrypsin (AAT); and
ii. if the concentration of circulating polymerized AAT is found to be at least 1 μg/mL, administering an AAT modulator, and if the concentration of circulating polymerized AAT is found to be less than 1 μg/mL, not administering an AAT modulator.

81. The method of claim 80, wherein the concentration of circulating polymerized AAT is determined using the method of any one of claims 1-32.

82. The method of claim 80 or 81, wherein the patient has a Z mutation in the AAT protein.

83. The method of claim 82, wherein the patient is heterozygous for the Z mutation.

84. The method of claim 83, wherein the patient is heterozygous for the Z mutation, and has an additional mutation in AAT associated with AATD.

85. The method of claim 82, wherein the patient has a PiZZ genotype.

86. The method of any one of claims 80-85, wherein the AAT modulator is

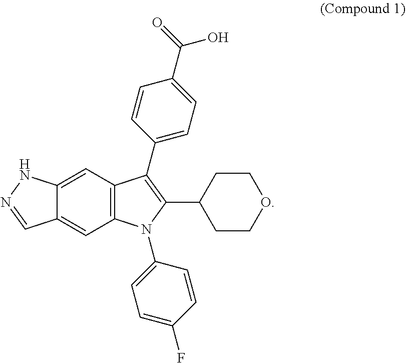 [SMILES: O=C(O)c1ccc(-c2c(C3CCOCC3)n(-c3ccc(F)cc3)c3cc4cn[nH]c4cc23)cc1]

87. A method for measuring the efficacy of an alpha-1 antitrypsin (AAT) modulator, wherein the method comprises:
i. administering the AAT modulator to a patient with alpha-1 antitrypsin deficiency (AATD); and
ii. measuring the change in the concentration of circulating polymerized AAT in the patient.

88. The method of claim 87, wherein the change in the concentration of circulating polymerized AAT is determined after administering the AAT modulator.

89. The method of claim 87 or 88, wherein the change in the concentration of circulating polymerized AAT is determined by obtaining a first measurement of the concentration of circulating polymerized AAT shortly before administering the AAT modulator, and obtaining a second measurement of the concentration of polymerized AAT shortly after administering the AAT modulator.

90. The method of claim 89, wherein the first measurement is obtained within 3 months of administering the AAT modulator.

91. The method of claim 89 or 90, wherein the second measurement is obtained within one month of administering the AAT modulator.

92. The method of any one of claims 89-91, wherein the second measurement is obtained within one week of administering the AAT modulator.

93. The method of any one of claims 87-92, wherein the concentration of circulating polymerized AAT is measured using the method of any one of claims 1-32.

94. A method for determining an efficacious dosing regimen of an AAT modulator for treating alpha-1 antitrypsin deficiency (AATD) in a patient, wherein the method comprises measuring the change in the concentration of circulating polymerized AAT in the patient.

95. The method of claim 94, wherein the method comprises measuring the change in the concentration of circulating polymerized AAT after administering the AAT modulator.

96. The method of claim 94 or 95, wherein the method comprises measuring the concentration of circulating polymerized AAT before and after administering the AAT modulator.

97. The method of any one of claims 94-96, wherein the dosage of the AAT modulator is increased if the concentration of circulating polymerized AAT is found to be above about 2 μg/mL.

98. The method of any one of claims 94-97, wherein the dosage of the AAT modulator is increased if the concentration of circulating polymerized AAT is found to be above about 1 μg/mL.
